(19) United States
(12) Patent Application Publication
(10) Pub. No.: US 2009/0124682 A1
(43) Pub. Date: May 14, 2009

(54) Indan-Amide Derivatives with Glycogen Phosphorylase Inhibitory Activity

(76) Inventors: Alan Martin Birch, Cheshire (GB); Craig Johnstone, Cheshire (GB); Alleyn Thomas Plowright, Cheshire (GB); Iain Simpson, Cheshire (GB); Paul Robert Owen Whittamore, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 11/883,770

(22) PCT Filed: Feb. 2, 2006
(86) PCT No.: PCT/GB06/00347

§ 371 Date: Aug. 6, 2007

(30) Foreign Application Priority Data
Feb. 5, 2005 (GB) 0502467.4
Feb. 5, 2005 (GB) 0502468.2

(51) Int. Cl. A61K 31/40; C07D 209/42; A61P 3/10; A61P 3/04; A61P 9/10

(52) U.S. Cl. 514/419; 548/492

(57) ABSTRACT

A compound of the formula (1) or a pharmaceutically-acceptable salt: (A chemical formula should be inserted here—please see paper copy enclosed herewith) (1) possess glycogen phosphorylase inhibitory activity and accordingly have value in the treatment of disease states associated with increased glycogen phosphorylase activity. Processes for the manufacture of compounds and pharmaceutical compositions containing them are described.

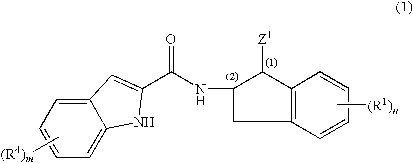

The present invention relates to indan amide derivatives, pharmaceutically acceptable salts and in-vivo hydrolysable esters thereof. These heterocyclic amide possess glycogen phosphorylase inhibitory activity and accordingly have value in the treatment of disease states associated with increased glycogen phosphorylase activity and thus are potentially useful in methods of treatment of a warm-blooded animal such as man. The invention also relates to processes for the manufacture of said heterocyclic amide derivatives, to pharmaceutical compositions containing them and to their use in the manufacture of medicaments to inhibit glycogen phosphorylase activity in a warm-blooded animal such as man.

The liver is the major organ regulating glycaemia in the post-absorptive state. Additionally, although having a smaller role in the contribution to post-prandial blood glucose levels, the response of the liver to exogenous sources of plasma glucose is key to an ability to maintain euglycaemia. An increased hepatic glucose output (HGO) is considered to play an important role in maintaining the elevated fasting plasma glucose (FPG) levels seen in type 2 diabetics; particularly those with a FPG>140 mg/dl (7.8 mM). (Weyer et al, (1999), J Clin Invest 104: 787-794; Clore & Blackgard (1994), Diabetes 43: 256-262; De Fronzo, R. A., et al, (1992) Diabetes Care 15; 318-355; Reaven, G. M. (1995) Diabetologia 38; 3-13).

Since current oral, anti-diabetic therapies fail to bring FPG levels to within the normal, non-diabetic range and since raised FPG (and glycHbAlc) levels are risk factors for both macro- (Charles, M. A. et al (1996) Lancet 348, 1657-1658; Coutinho, M. et al (1999) Diabetes Care 22; 233-240; Shaw, J. E. et al (2000) Diabetes Care 23, 34-39) and micro-vascular disease (DCCT Research Group (1993) New. Eng. J. Med. 329; 977-986); the reduction and normalisation of elevated FPG levels remains a treatment goal in type 2 DM.

It has been estimated that, after an overnight fast, 74% of HGO was derived from glycogenolysis with the remainder derived from gluconeogenic precursors (Hellerstein et al (1997) Am J Physiol, 272: E163). Glycogen phosphorylase is a key enzyme in the generation by glycogenolysis of glucose-1-phosphate, and hence glucose in liver and also in other tissues such as muscle and neuronal tissue.

Liver glycogen phosphorylase a activity is elevated in diabetic animal models including the db/db mouse and the fa/fa rat (Aiston S et al (2000). Diabetalogia 43, 589-597).

Inhibition of hepatic glycogen phosphorylase with chloroindole inhibitors (CP91149 and CP320626) has been shown to reduce both glucagon stimulated glycogenolysis and glucose output in hepatocytes (Hoover et al (1998) J Med Chem 41, 2934-8; Martin et al (1998) PNAS 95, 1776-81). Additionally, plasma glucose concentration is reduced, in a dose related manner, db/db and ob/ob mice following treatment with these compounds.

Studies in conscious dogs with glucagon challenge in the absence and presence of another glycogen phosphorylase inhibitor, Bay K 3401, also show the potential utility of such agents where there is elevated circulating levels of glucagon, as in both Type 1 and Type 2 diabetes. In the presence of Bay R 3401, hepatic glucose output and arterial plasma glucose following a glucagon challenge were reduced significantly (Shiota et al, (1997), Am J Physiol, 273: E868).

The indan amides of the present invention possess glycogen phosphorylase inhibitory activity and accordingly are expected to be of use in the treatment of type 2 diabetes, insulin resistance, syndrome X, hyperinsulinaemia, hyperglucagonaemia, cardiac ischaemia and obesity, particularly type 2 diabetes.

The compounds of the present invention have favourable physical properties, for examples good solubility.

According to one aspect of the present invention there is provided a compound of formula (1):

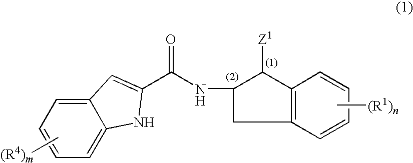

wherein:
n is 0, 1 or 2;
m is 0, 1 or 2;
R¹ is independently selected from halo, nitro, cyano, hydroxy, carboxy, carbamoyl, N-(1-4C)alkylcarbamoyl, N,N-((1-4C)alkyl)₂-carbamoyl, sulphamoyl, N-(1-4C)alkylsulphamoyl, N,N-((1-4C)allyl)₂sulphamoyl, (1-4C)alkylS(O)_{b} (wherein b is 0, 1, or 2), —OS(O)₂(1-4C)alkyl, (1-4C)alkyl, (2-4C)alkenyl, (2-4C)alkynyl, (1-4C)alkoxy, (1-4C)alkanoyl, (1-4C)alkanoyloxy, hydroxy(1-4C)alkyl, fluoromethyl, difluoromethyl, trifluoromethyl, trifluoromethoxy and —NHSO₂(1-4C)alkyl;
or, when n is 2, the two R¹ groups, together with the carbon atoms to which they are attached, may form a 4 to 7 membered saturated ring, optionally containing 1 or 2 heteroatoms independently selected from O, S and N, and optionally being substituted by one or two methyl groups;
R⁴ is independently selected from halo, nitro, cyano, hydroxy, fluoromethyl, difluoromethyl, trifluoromethyl, trifluoromethoxy, carboxy, carbamoyl, (1-4C)alkyl, (2-4C)alkenyl, (2-4C)alkynyl, (1-4C)alkoxy and (1-4C)alkanoyl;
Z¹ is either
a) of the formula —Y—COOH wherein Y is (1-6C)alkylene or (3-6C)cycloalkylene; or
b) of the formula —Y—COOH; wherein Y is (1-6C)alkylene which is:
i) interrupted by one heteroatom selected from —N(R⁷)—, —O—, —S—, —SO— and —SO₂— (provided that the heteroatom is not adjacent to the carboxy group and wherein R⁷ is hydrogen, (1-4C)alkyl, (1-4C)alkanoyl or (1-4C)alkylsulphonyl); and/or
ii) substituted on carbon by 1 or 2 substituents independently selected from cyano, oxo, hydroxyl, (1-3C)alkoxy, (1-3C)alkanoyl, (1-3C)alkoxy(2-3C)alkoxy, hydroxy(1-3C)alkyl, hydroxy(2-3C)alkoxy, (3-6C)cycloalkyl, (3-6C)cycloalkyl(1-3C)alkyl, (3-6C)cycloalkyloxy, (3-6C)cycloalkyl(1-3C)alkoxy, (1-3C)alkylS(O), (wherein c is 0, 1 or 2), —CON(R²)R³, —N(R²)COR³, —SO₂N(R²)R³ and —N(R²)SO₂R³ wherein R² and R³ are independently selected from hydrogen and (1-3C)alkyl;
or when the alkylene group is interrupted by one heteroatom it may also be optionally substituted on a carbon by 2 substituents which together with the carbon atom to which they are attached form a (3-6C)cycloalkyl ring;
   or a pharmaceutically acceptable salt thereof.

In another aspect, the invention relates to compounds of formula (1) as hereinabove defined or to a pro-drug thereof. Suitable examples of pro-drugs of compounds of formula (1) are in-vivo hydrolysable esters of compounds of formula (1). Therefore in another aspect, the invention relates to compounds of formula (1) as hereinabove defined or to an in-vivo hydrolysable ester thereof.

It is to be understood that, insofar as certain of the compounds of formula (1) defined above may exist in optically active or racemic forms by virtue of one or more asymmetric carbon atoms, the invention includes in its definition any such optically active or racemic form which possesses glycogen phosphorylase inhibition activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

Within the present invention it is to be understood that a compound of the formula (1) or a salt thereof may exhibit the phenomenon of tautomerism and that the formulae drawings within this specification can represent only one of the possible tautomeric forms. It is to be understood that the invention encompasses any tautomeric form which has glycogen phosphorylase inhibition activity and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein.

It is also to be understood that certain compounds of the formula (1) and salts thereof can exist in solvated as well as unsolvated forms such as, for example, hydrated forms. It is to be understood that the invention encompasses all such solvated forms which have glycogen phosphorylase inhibition activity.

It is also to be understood that certain compounds of the formula (1) may exhibit polymorphism, and that the invention encompasses all such forms which possess glycogen phosphorylase inhibition activity.

The present invention relates to the compounds of formula (1) as hereinbefore defined as well as to the salts thereof. Salts for use in pharmaceutical compositions will be pharmaceutically acceptable salts, but other salts may be useful in the production of the compounds of formula (1) and their pharmaceutically acceptable salts. Pharmaceutically acceptable salts of the invention may, for example, include acid addition salts of the compounds of formula (1) as hereinbefore defined which are sufficiently basic to form such salts. Such acid addition salts include for example salts with inorganic or organic acids affording pharmaceutically acceptable anions such as with hydrogen halides (especially hydrochloric or hydrobromic acid, of which hydrochloric acid is particularly preferred) or with sulphuric or phosphoric acid, or with trifluoroacetic, citric or maleic acid. Suitable salts include hydrochlorides, hydrobromides, phosphates, sulphates, hydrogen sulphates, alkylsulphonates, arylsulphonates, acetates, benzoates, citrates, maleates, fumarates, succinates, lactates and tartrates. In addition where the compounds of formula (1) are sufficiently acidic, pharmaceutically acceptable salts may be formed with an inorganic or organic base which affords a pharmaceutically acceptable cation. Such salts with inorganic or organic bases include for example an alkali metal salt, such as a sodium or potassium salt, an alkaline earth metal salt such as a calcium or magnesium salt, an ammonium salt or for example a salt with methylamine, dimethylamine, trimethylamine, piperidine, morpholine or tris-(2-hydroxyethyl)amine.

The compounds of the invention may be administered in the form of a pro-drug which is broken down in the human or animal body to give a compound of the invention. A prodrug may be used to alter or improve the physical and/or pharmacokinetic profile of the parent compound and can be formed when the parent compound contains a suitable group or substituent which can be derivatised to form a prodrug. Examples of pro-drugs include in-vivo hydrolysable esters of a compound of the invention or a pharmaceutically-acceptable salt thereof.

An in-vivo hydrolysable ester of a compound of formula (1) containing carboxy or hydroxy group is, for example. A pharmaceutically acceptable ester which is cleaved in the human or animal body to produce the parent acid or alcohol.

Suitable pharmaceutically acceptable esters for carboxy include (1-6C)alkoxymethyl esters for example methoxymethyl, (1-6C)alkanoyloxymethyl esters for example pivaloyloxymethyl, phthalidyl esters, (3-8C)cycloalkoxycarbonyloxy(1-6C)alkyl esters for example 1-cyclohexylcarbonyloxyethyl; 1,3-dioxolen-2-onylmethyl esters for example 5-methyl-1,3-dioxolen-2-onylmethyl; and (1-6C)alkoxycarbonyloxyethyl esters for example 1-methoxycarbonyloxyethyl and may be formed at any carboxy group in the compounds of this invention.

Suitable pharmaceutically-acceptable esters for hydroxy include inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxymethoxy. A selection of in-vivo hydrolysable ester forming groups for hydroxy include (1-10C)alkanoyl, for example acetyl; benzoyl; phenylacetyl; substituted benzoyl and phenylacetyl, (1-10C)alkoxycarbonyl (to give alkyl carbonate esters), for example ethoxycarbonyl; di-((1-4C))alkylcarbamoyl and N-(di-((1-4C))alkylaminoethyl)-N-((1-4C))alkylcarbamoyl (to give carbamates); di-((1-4C))alkylaminoacetyl and carboxyacetyl. Examples of ring substituents on phenylacetyl and benzoyl include aminomethyl, ((1-4C))alkylaminomethyl and di-(((1-4C))alkyl)aminomethyl, and morpholino or piperazino linked from a ring nitrogen atom via a methylene linking group to the 3- or 4-position of the benzoyl ring. Other interesting in-vivo hyrolysable esters include, for example, R^{A}C(O)O(1-6C)alkyl-CO—, wherein R^{A} is for example, benzyloxy-((1-4C))alkyl, or phenyl). Suitable substituents on a phenyl group in such esters include, for example, 4-((1-4C)alkyl)piperazino-(1-4C)alkyl, piperazino-(1-4C)alkyl and morpholino-(C₁-C₄)alkyl.

In this specification the generic term “alkyl” includes both straight-chain and branched-chain alkyl groups. However references to individual alkyl groups such as “propyl” are specific for the straight chain version only and references to individual branched-chain alkyl groups such as t-butyl are specific for the branched chain version only. For example, “(1-4C)alkyl” includes methyl, ethyl, propyl, isopropyl and t-butyl and examples of “(1-6C)alkyl” include the examples of “(1-4C)alkyl” and additionally pentyl, 2,3-dimethylpropyl, 3-methylbutyl and hexyl. An analogous convention applies to other generic terms, for example “(2-4C)alkenyl” includes vinyl, allyl and 1-propenyl and examples of “(2-6C)alkenyl” include the examples of “(2-4C)alkenyl” and additionally 1-butenyl, 2-butenyl, 3-butenyl, 2-methylbut-2-enyl, 3-methylbut-1-enyl, 1-pentenyl, 3-pentenyl and 4-hexenyl. Examples of “(2-4C)alkynyl” includes ethynyl, 1-propynyl and 2-propynyl and examples of “(2-6C)alkynyl” include the examples of “(2-4C)alkynyl” and additionally 3-butynyl, 2-pentynyl and 1-methylpent-2-ynyl.

The term “hydroxy(1-4C)alkyl” includes hydroxymethyl, hydroxyethyl, hydroxypropyl, hydroxyisopropyl and hydroxybutyl. The term “hydroxyethyl” includes 1-hydroxyethyl and 2-hydroxyethyl. The term “hydroxypropyl” includes 1-hydroxypropyl, 2-hydroxypropyl and 3-hydroxypropyl and an analogous convention applies to terms such as hydroxybutyl. The term “dihydroxy(1-4C)alkyl” includes dihydroxyethyl, dihydroxypropyl, dihydroxyisopropyl and dihydroxybutyl. The term “dihydroxypropyl” includes 1,2-dihydroxypropyl and 1,3-dihydroxypropyl. An analogous convention applies to terms such as dihydroxyisopropyl and dihydroxybutyl.

The term “halo” refers to fluoro, chloro, bromo and iodo. The term “dihalo(1-4C)alkyl” includes difluoromethyl and dichloromethyl. The term “trihalo(1-4C)alkyl” includes trifluoromethyl.

Examples of “(1-3C)alkoxy”, “(1-4C)alkoxy” and “—O(1-4C)alkyl” include methoxy, ethoxy, propoxy and isopropoxy. Examples of “(1-6C)alkoxy” include the examples of “(1-4C)alkoxy” and additionally butyloxy, t-butyloxy, pentoxy and 1,2-(methyl)₂propoxy. Examples of “hydroxy(2-3C)alkoxy” include 1-hydroxyethoxy, 1-hydroxypropoxy and 2-hydroxypropoxy; Examples of (1-3C)alkoxy(2-3C)alkoxy include methoxyethoxy, ethoxyethoxy and methoxypropoxy; Examples of “(1-3C)alkanoyl” and “(1-4C)alkanoyl” include formyl, acetyl and propionyl. Examples of “(1-6C)alkanoyl” include the example of “(1-4C)alkanoyl” and additionally butanoyl, pentanoyl, hexanoyl and 1,2-(methyl)₂propionyl. Examples of “(1-4C)alkanoyloxy” include formyloxy, acetoxy and propionoxy. Examples of “(1-6C)alkanoyloxy” include the examples of “(1-4C)alkanoyloxy” and additionally butanoyloxy, pentanoyloxy, hexanoyloxy and 1,2-(methyl)₂propionyloxy Examples of “N-((1-4C)alkyl)carbamoyl” are methylcarbamoyl and ethylcarbamoyl. Examples of “N,N-((1-4C)alkyl)₂-carbamoyl” are N,N-(methyl)₂-carbamoyl, N,N-(ethyl)₂-carbamoyl and N-methyl-N-ethylcarbamoyl. Examples of “N-((1-4C)alkyl)sulphamoyl” are N-(methyl)sulphamoyl and N-(ethyl)sulphamoyl. Examples of “N,N-((1-4C)alkyl)₂sulphamoyl” are N,N-(methyl)₂sulphamoyl, N,N-(ethyl)₂sulphamoyl and N-(methyl)-N-(ethyl)sulphamoyl. Examples of —NHSO₂(1-4C)alkyl are methylsulfonylamino, ethylsulfonylamino, propylsulfonylamino, isopropylsulfonylamino and tert-butylsulfonylamino.

Examples of “(1-4C)alkylS(O)_{b} (wherein b is 0, 1 or 2)”, “(1-4C)alkylS(O)_{c} (wherein c is 0 to 2)”, “(1-3C)alkylS(O), (wherein c is 0 to 2)” and “(1-4C)alkylS(O)_{d} (wherein d is 0 to 2)”, independently include methylthio, ethylthio, propylthio, methanesulphinyl, ethanesulphinyl, propanesulphinyl, mesyl, ethanesulphonyl, propanesulphonyl and isopropanesulphonyl. Examples of “(1-4C)alkylS(O)_{b}(1-4C)alkyl-” (wherein b is 0, 1 or 2)” include methylsulfonylmethyl, methylsulfinylmethyl, methylthiomethyl, ethylsulfonylmethyl, ethylsulfinylmethyl and ethylthiomethyl. Examples of “(1-4C)alkylsulfonyl” include mesyl, ethanesulphonyl, propanesulphonyl and isopropanesulphonyl. Examples of “—OSO₂(1-4C)alkyl” include methylsulfonyloxy, ethylsulfonyloxy, propylsulfonyloxy, isopropylsulfonyloxy and tert-butylsulfonyloxy.

Examples of “(3-6C)cycloalkyl” include cyclopropyl, cyclobutyl, cyclopentyl and cyclohexyl. Examples of “(3-6C)cycloalkyl(1-3C)alkyl” include cyclopropylmethyl, cyclobutylmethyl, cyclopentylmethyl and cyclohexylmethyl. Examples of “(3-6C)cycloalkoxy” include cyclopropyloxy, cyclobutyloxy, cyclopentyloxy and cyclohexyloxy. Examples of “(3-6C)cycloalkyl(1-3C)alkoxy” include cyclopropylmethoxy, cyclobutylmethoxy, cyclopentylmethoxy and cyclohexylmethoxy.

Within this specification composite terms are used to describe groups comprising more that one functionality such as -(1-4C)alkylSO₂(1-4C)alkyl. Such terms are to be interpreted in accordance with the meaning which is understood by a person skilled in the art for each component part.

Examples of (3-6C)cycloalkylene groups include cycloprop-1-ylene, cyclobut-1-ylene and cyclopent-1-ylene.

For the avoidance of doubt it is to be understood that where in this specification a group is qualified by ‘hereinbefore defined’ or ‘defined hereinbefore’ the said group encompasses the first occurring and broadest definition as well as each and all of the particular definitions for that group.

It is to be understood that where substituents contain two substituents on an alkyl chain, in which both are linked by a heteroatom (for example two alkoxy substituents), then these two substituents are not substituents on the same carbon atom of the alkyl chain.

It is to be understood that optional substituents on any group may be attached to any available atom as appropriate unless otherwise specified, including heteroatoms provided that they are not thereby quaternised. Therefore, hydroxy substituted (1-6C)alkyl includes hydroxymethyl, 1-hydroxyethyl, 2-hydroxyethyl and 3-hydroxypropyl.

For the avoidance of doubt, where Z¹=—Y—COOH wherein Y is (1-6C)alkylene which is interrupted by one heteroatom (and optionally also substituted), the (1-6C)alkylene group may be branched and any optional substituents may be on the branch, such that this definition of Z¹ includes structures such as that shown below (wherein Y is ethylene substituted by methoxy).

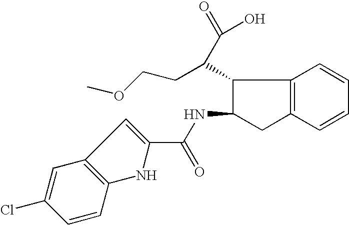

Where optional substituents are chosen from “0, 1 or 2” groups it is to be understood that this definition includes all substituents being chosen from one of the specified groups or the substituents being chosen from two or more of the specified groups.

Examples of (1-6C)allylene groups interrupted by a heteroatom selected from nitrogen, oxygen and sulphur include the diradicals —CH₂XCH₂—, —CH₂XCH₂CH₂—, —CH₂CH₂XCH₂—, —CH(R^{a})XCH₂—, —CH(R^{a})XCH₂CH₂—, —CH(R^{e})CH₂×CH₂—, —CH₂CH(R^{a})XCH₂—, —CH₂CH₂XCH(R^{a})—, —CH₂XCH(R^{a})CH₂—, —CH₂XCH₂CH(R^{a})— [wherein X is selected from —O—, —S—, —SO—, —SO₂— and —N(R^{c}) (wherein R^{c} is selected from methyl, ethyl, formyl, acetyl and methanesulfonyl) and R^{a} is selected from methyl and ethyl]. The right side of the linker is attached to the COOH group in Z¹.

Further examples of (1-6C)alkylene groups interrupted by a heteroatom include —CH₂XCH₂—, —CH₂XCH₂CH₂—, —CH₂CH₂XCH₂, —CH(R^{f})XCH₂—, —CH(R^{f})XCH₂CH₂—, —CH(R^{f})CH₂XCH₂—, —CH₂CH(R^{f})XCH₂—, —CH₂CH₂XCH(R^{f})—, —CH₂×CH(R^{f})CH₂—, —CH₂XCH(R^{f})—, —CH₂XCR^{f}₂—, —CH₂XCH₂CH₂CH₂—, —CH(CH₂XCH₂CH₃)—, —CH(CH₂XCH₃)—, —CH(CH₂CH₂XCH₃)—, —CH(CH₂CH₂XCH₂CH₃)—, —CH(CH₂CH₂CH₂XCH₃)—, —CH(CH₂XCH₂CH₃)CH₂—, —CH(CH₂XCH₃)CH₂—, —CH(CH₂CH₂XCH₃)CH₂—, —CH(CH₂CH₂XCH₂CH₃)CH₂— and —CH(CH₂CH₂CH₂XCH₃)CH₂—, [wherein X is as defined above and in particular is selected from —O—, —S— and —SO₂—, and R^{f} is selected from methyl and ethyl]. The right side of the linker is bonded to the COOH group in Z¹.

Examples of (1-6C)alkylene groups include the diradicals methylene, ethylene, propylene, butylene, —CH(Me)—, —CH(Et)-, —C(Me)₂-, —CH₂CH(Me)—, —CH₂CH(Et)- and —CH₂C(Me)₂-. The right side of the linker is bonded to the COOH group in Z¹.

Particular values of Y, R¹, R⁴, n and m are as follows. Such values may be used where appropriate with any of the definitions, claims, aspects or embodiments defined hereinbefore or hereinafter.

In one embodiment of the invention are provided compounds of formula (1), in an alternative embodiment are provided pharmaceutically-acceptable salts of compounds of formula (1), in a further alternative embodiment are provided in-vivo hydrolysable esters of compounds of formula (1), and in a further alternative embodiment are provided pharmaceutically-acceptable salts of in-vivo hydrolysable esters of compounds of formula (1).

In a further alternative embodiment are provided pro-drugs of compounds of formula (1) and in a still further alternative embodiment are provided pharmaceutically-acceptable salts of pro-drugs of compounds of formula (1).

### Particular Values for m

i) In one aspect of the present invention m is 1 or 2.
ii) In another aspect of the invention m is 1.
iii) In another aspect of the invention m is 0.

### Particular Values for R⁴

i) In one aspect of the present invention R⁴ is selected from halo, cyano, hydroxy, fluoromethyl, difluoromethyl and trifluoromethyl.
ii) In another aspect of the invention R⁴ is halo.
iii) In yet another aspect R⁴ is selected from chloro and bromo.
iv) In a further aspect R⁴ is selected from chloro, fluoro and methyl.
v) In a further aspect R⁴ is selected from chloro and fluoro
vi) More particularly R⁴ is chloro and if m is 1 it is in the 5-position.

### Particular Values for n

i) In one aspect of the invention n is 0 or 1.
ii) In one aspect preferably n is 1.
iii) In another aspect, preferably n is 0.
   Particular Values for R¹ when n is 2
i) When n is 2, and the two R¹ groups, together with the carbon atoms to which they are attached, form a 4 to 7 membered saturated ring, optionally containing 1 or 2 heteroatoms independently selected from O, S and N, conveniently such a ring is a 5 or 6 membered ring.
ii) In one embodiment such a 5 or 6 membered ring contains two O atoms (i.e. a cyclic acetal).
iii) When the two R¹ groups together form such a cyclic acetal, in one aspect, it is not substituted.
iv) Most particularly, the two R¹ groups together are the group —O—CH₂—O—.

### Particular Values for R¹

i) In another aspect of the present invention R¹ is selected from halo, nitro, cyano, hydroxy, fluoromethyl, difluoromethyl, trifluoromethyl and (1-4C)alkoxy.
ii) In a further aspect R¹ is selected from halo, nitro, cyano, hydroxy, fluoromethyl, difluoromethyl, trifluoromethyl, —S(O)_{b}(1-4C)alkyl (wherein b is 0, 1 or 2), —OS(O)₂(1-4C)alkyl, (1-4C)alkyl and (1-4C)alkoxy.
iii) In a further aspect R¹ is selected from halo, nitro, cyano, hydroxy, fluoromethyl, difluoromethyl, trifluoromethyl, —S(O)_{b}Me (wherein b is 0, 1 or 2), —OS(O)₂Me, methyl and methoxy.
iv) In a further aspect, R¹ is (1-4C)alkyl.
v) Particularly R¹ is selected from halo and (1-4C)alkoxy.
vi) In another embodiment preferably R¹ is selected from fluoro, chloro, methyl, ethyl, methoxy and —O—CH₂—O—.

In one aspect Y is selected from option a).

In another aspect, Y is selected from option b), particularly b)i).

### Particular Values for Y for Option a)

i) In one aspect Y is (3-6C)cycloalkylene.
ii) In another aspect Y is cyclopropylene, methylenecycloprop-1-yl, methylenecyclobut-1-yl or methylenecyclopent-1-yl.
iii) In another aspect Y is (1-6C)alkylene.
iv) In another aspect Y is selected methylene, ethylene, propylene, butylene, —CH(Me)—, —CH(Et)-, —C(Me)₂-, —CH₂CH(Me)—, —CH₂CH(Et)- and —CH₂C(Me)₂-.
v) In yet another aspect Y is selected from methylene and ethylene.

### Particular Values for Y for Option b)

vi) Particular values for Y include —CH₂XCH₂—, —CH₂XCH₂CH₂—, —CH₂CH₂XCH₂, —CH(R^{a})XCH₂—, —CH(R^{a})XCH₂CH₂—, —CH(R^{a})CH₂XCH₂—, —CH₂CH(R^{a})XCH₂—, —CH₂CH₂XCH(R^{a})—, —CH₂XCH(R^{a})CH₂—, —CH₂×CH₂CH(R^{b})— [wherein X is selected from —O—, —S—, —SO—, —SO₂ and —N(R^{c}) (wherein R¹ is selected from methyl, ethyl, formyl, acetyl, methanesulfonyl) and R^{a} is selected from methyl and ethyl and R^{b} is selected from methyl, ethyl, methoxy and ethoxy], —CH₂C(Me)₂OCH₂—, —CH₂CH₂OC(Me)₂-, —CH₂C(Me)₂CH₂—, —CH₂OCH₂C(Me)₂-, —CH(R^{d})— (wherein R^{d} is selected from cyclopropyl, cyclopropylmethyl, methoxy, ethoxy, methoxyethyl, cyclopropylmethoxy, methoxyethoxy and cyano), —CH₂CH(R^{e})— (wherein R^{e} is selected from cyclopropyl, cyclopropylmethyl, methoxy, ethoxy, cyclopropylmethoxy, methoxyethoxy, cyano, methylthio, methylsulphinyl, methylsulphonyl, aminosulphonyl, N-methylaminosulphonyl, N,N-di-methylaminosulphonyl, methanesulphonamido, N-methyl-methanesulphonamido, acetyl, acetamido, N-methylacetamido, carbamoyl, N-methylcarbamoyl and N,N-dimethylcarbamoyl), methylenecycloprop-1-yloxymethyl (—CH₂C(CH₂CH₂)OCH₂—), ethyleneoxycycloprop-1-yl, methyleneoxycycloprop-1-ylmethyl and methyleneoxymethylcyclprop-1-yl.
vii) Further particular values for Y include —CH₂XCH₂—, —CH₂XCH₂CH₂—, —CH₂CH₂XCH₂, —CH(R)XCH₂—, —CH(R^{f})XCH₂CH₂—, —CH(R^{f})CH₂XCH₂—, —CH₂CH(R^{f})XCH₂—, —CH₂CH₂XCH(R^{f})—, —CH₂XCH(R^{f})CH₂—, —CH₂XCH(R^{f})—, —CH₂XCR^{f}₂—, —CH₂XCH₂CH₂CH₂— [wherein X is selected from —O—, —S— and —SO₂₋ and R^{f} is selected from methyl and ethyl], —CH₂—, —CH₂CH₂—, —CH₂CH₂CH₂—, —CH₂CH(Me)—, —CH(R^{g})— and —CH(R^{g})CH₂— [wherein R^{g} is selected from methoxymethyl, ethoxyethyl, methoxyethyl, ethoxymethyl, methoxypropyl, cyclopropylmethyl, isopropylmethyl, ethyl and propyl]
viii) Further particular values for Y include —CH₂—, —CH(CH₃)—, —CH₂OCH₂—, —CH₂OCH(CH₃)— and —CH(CH₂CH₂OCH₃)—

Particular classes of compounds of the present invention are disclosed in Tables A, B and C using combinations of the definitions described hereinabove. For example, ‘i’ in the column headed R¹ in the table refers to definition (i) given for R¹ hereinabove and ‘I’ refers to the first definition given for the variables in the compound of formula (1) at the beginning of the description. It will be understood that for the definition of Y, “a)I” refers to the first definition for the variable under option a) in the compound of formula (1) at the beginning of the description and that a similar convention applies to “b)i”.

TABLE A

| Class | R¹ | n | Y | R⁴ | m |
|---|---|---|---|---|---|
| 1 | i | I | a)I | v | i |
| 2 | ii | i | i | vi | ii |
| 3 | iii | i | iii | vi | ii |
| 4 | iv | i | iv | vi | ii |
| 5 | v | i | v | vi | ii |
| 6 | — | iii | v | vi | ii |

TABLE B

| Class | R¹ | n | Y | R⁴ | m |
|---|---|---|---|---|---|
| 1 | i | I | b)i | v | i |
| 2 | ii | i | b)i | — | iii |
| 3 | iii | i | b)i | vi | ii |
| 4 | iv | i | vi | vi | ii |
| 5 | v | i | vi | vi | ii |
| 6 | — | iii | vi | vi | ii |

Further particular compounds of the invention are those defined in Table C:

TABLE C

| Class | R⁴ | m | n | Y |
|---|---|---|---|---|
| 1 | v | i | iii | vi |
| 2 | v | i | iii | vii |
| 3 | v | i | iii | viii |

In one aspect of the invention, the compound of formula (1) is a compound of formula (1A):

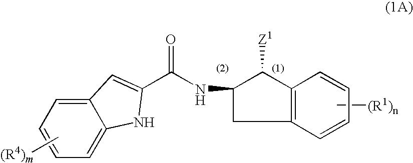

It will be understood that the particular values, aspects and embodiments described above for compounds of formula (1) also apply to compounds of formula (1A).

Particular compounds of the invention are each of the Examples or a pharmaceutically-acceptable salt thereof, each of which provides a further independent aspect of the invention. In a further aspect of the invention there is provided any two or more of the Examples or a pharmaceutically-acceptable salt thereof.

Further particular compounds of the invention comprises any one or more of the following (or their pharmaceutically-acceptable salts):
[((1R,2R)-2-{[(5-chloro-1H-indol-2-yl)carbonyl]amino}-2,3-dihydro-1H-inden-1-yl)methoxy]acetic acid;
[((1R,2R)-2-{[(5-fluoro-1H-indol-2-yl)carbonyl]amino}-2,3-dihydro-1H-inden-1-yl)methoxy]acetic acid;
(2R/S)-[((1R,2R)-2-{[(5-chloro-1H-indol-2-yl)carbonyl]amino}-2,3-dihydro-1H-inden-1-yl)methoxy]propanoic acid;
(2R/S)-[((1R,2R)-2-{[(5-fluoro-1H-indol-2-yl)carbonyl]amino}-2,3-dihydro-1H-inden-1-yl)methoxy]propanoic acid;
((1R,2R)-2-{[(5-fluoro-1H-indol-2-yl)carbonyl]amino}-2,3-dihydro-1H-inden-1-yl)acetic acid;
((1R,2R)-2-{[(5-chloro-1H-indol-2-yl)carbonyl]amino}-2,3-dihydro-1H-inden-1-yl)acetic acid
((1R,2R)-2-{[(5,6-difluoro-1H-indol-2-yl)carbonyl]amino}-2,3-dihydro-1H-inden-1-yl)acetic acid;
{(1R,2R)-2-[(5-chloro-7-fluoro-1H-indole-2-carbonyl)-amino]-indan-1-yl}-acetic acid;
(2R)-2-{(1R,2R)-2-[(5-chloro-1H-indole-2-carbonyl)-amino]-indan-1-yl}-4-methoxy-butyric acid;
(2S)-2-{(1R,2R)-2-[(5-chloro-1H-indole-2-carbonyl)-amino]-indan-1-yl}-4-methoxy-butyric acid;
(2R)-2-{(1R,2R)-2-[(5-chloro-1H-indole-2-carbonyl)-amino]-indan-1-yl}-propionic acid; and
(2S)-2-{(1R,2R)-2-[(5-chloro-1H-indole-2-carbonyl)-amino]-indan-1-yl}-propionic acid.

Another aspect of the present invention provides a process for preparing a compound of formula (1) or a pharmaceutically acceptable salt or an in-vivo hydrolysable ester thereof which process (wherein Z₁, Y, R¹, R⁴, m, and n are, unless otherwise specified, as defined in formula (1)) comprises of:

a) reacting an acid of the formula (2):

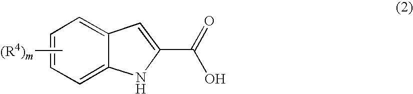

or an activated derivative thereof; with an amine of formula (3):

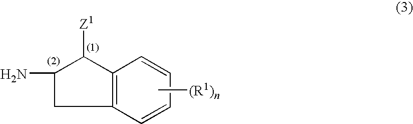

and thereafter if necessary:
i) converting a compound of the formula (1) into another compound of the formula (1);
ii) removing any protecting groups;
iii) forming a pharmaceutically acceptable salt or in vivo hydrolysable ester.

Specific reaction conditions for the above reaction are as follows.

Process a) Acids of formula (2) and amines of formula (3) may be coupled together in the presence of a suitable coupling reagent. Standard peptide coupling reagents known in the art can be employed as suitable coupling reagents, or for example carbonyldiimidazole, 1-ethyl-3-(3-dimethylaminopropyl)carbodi-imide hydrochloride (EDCI) and dicyclohexyl-carbodiimide (DCCI), optionally in the presence of a catalyst such as 1-hydroxybenzotriazole, dimethylaminopyridine or 4-pyrrolidinopyridine, optionally in the presence of a base for example triethylamine, di-isopropylethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

The acids of formula (2) are commercially available or they are known compounds or they are prepared by processes known in the art.

Compounds of formula (3) are either known compounds, may be prepared by processes known in the art or may be prepared according to Scheme 1 to 6 or by the methods used in the specific examples:

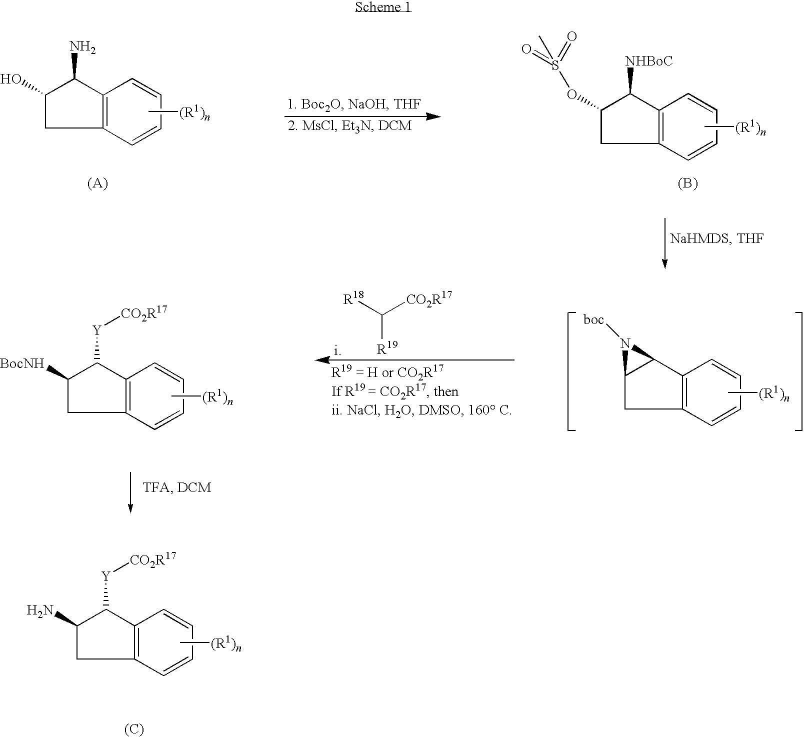

(where R¹⁷=(1-6)C alkyl and R¹⁸ is a variable related to Y—for example when Y is CHCH₃ then R¹⁸ is CH₃, or when Y is CH(OCH₃) then R¹⁸ is OCH₃). Compound A (where R¹ is hydrogen) is commercially available [(1R,2R)-(−)-trans-1-amino-2-indanol, Cas. Reg. No.:163061-73-2 or [(1S,2S)-(−)-trans-1-amino-2-indanol Cas. Reg. No.:13286-59-4]. Compounds of type B can be prepared by methods known in the literature, such as those shown above in Scheme 1. It will be appreciated that the process shown in Scheme 1 applies equally to the opposite enantiomers of compounds A, B and C to those shown. Compound (C) is then coupled to the appropriate acid (2) and the acid protecting group R¹⁷ is then removed by known methods in the art, for example, trifluoroacetic acid or potassium hydroxide.

Similarly, a process according to Scheme 2 may be used:

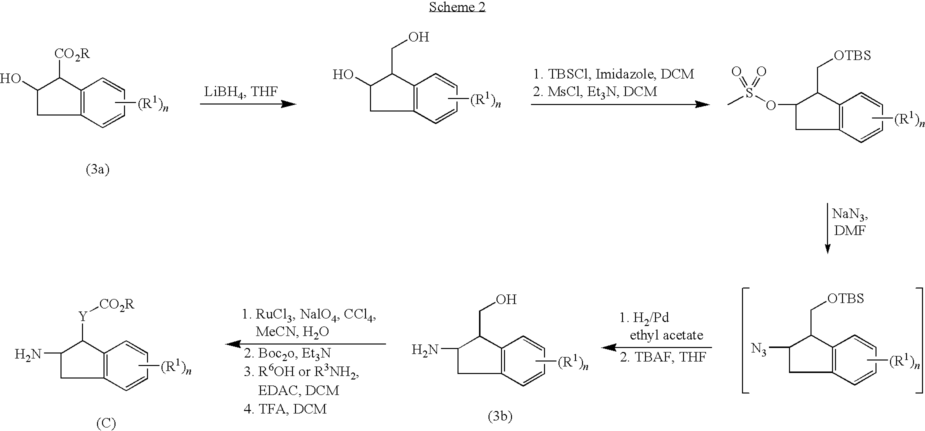

(where R⁹ is (1-6C)alkyl and R⁹ is a variable related to Y—for example if Y is CH₂C(O)NHCH₂ then R⁸ is CH₂CO₂R₉). (C) is then coupled to the appropriate acid (2) and the acid protecting group R¹ is then removed by well known methods in the art, for example, trifluoroacetic acid or potassium hydroxide.

Compounds of formula (3a) are commercially available or they are known compounds or they are prepared by processes known in the art. For example, starting from primary amines of formula (4), in which R is H or a suitable protecting group, R¹ may be introduced by acylation, (for example reacting with acetoxyacetic acid and 1-(3-dimethylaminopropyl)-3-ethyl-carbodiimide hydrochloride (EDAC)), alkylation, reductive alkylation, sulphonation or related processes, followed by O-deprotection when appropriate Alternatively, R¹ may be obtained by modification of functionality in groups previously thus introduced, by reduction, oxidation, hydrolysis (for example the conversion of an acetoxy group to a hydroxy group), nucleophilic displacement, amidation, or a related process, or a combination of these processes, followed by O-deprotection when appropriate. It will be appreciated that such modifications may include modifications which convert one compound of the formula (1) into another compound of the formula (1).

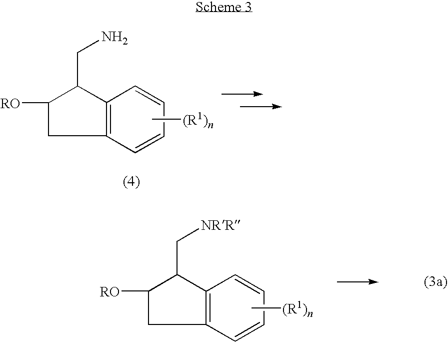

Amines of formula (3) may alternatively be obtained by applying the processes described for the preparation of compounds of formula (3a) to compounds of formula (5) in which W is NH₂ or a nitrogen atom with one or two suitable protecting groups.

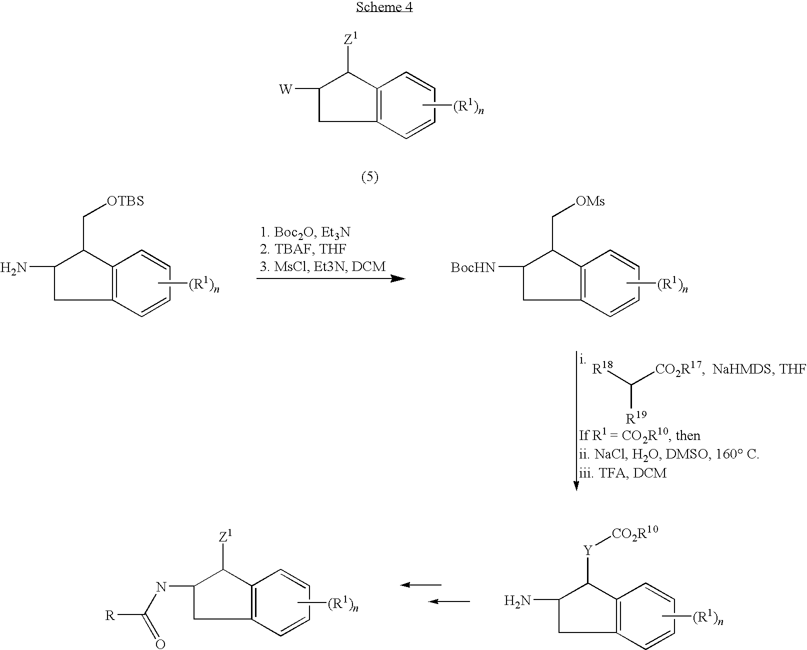

(wherein R¹ is hydrogen or CO₂R¹⁰; R¹⁰ is (1-6)C alkyl or an appropriately protected acid; and R¹¹ is a variable related to Y—for example when Y is CH₂CH(OCH₃) then R¹¹ is OCH₃) (C) is then coupled to the appropriate acid (2) and the acid protecting group R¹⁰ is then removed by well known methods in the art, for example, trifluoroacetic acid or potassium hydroxide.

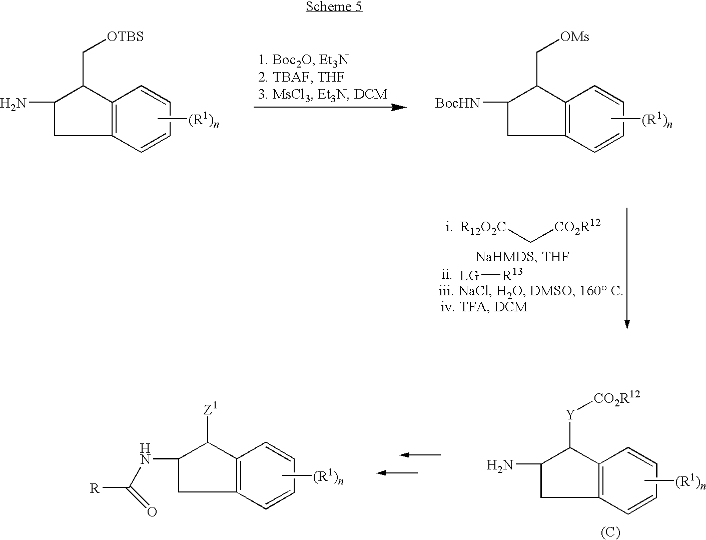

(where R¹² is independently (1-6C)alkyl or a carboxy-protecting group and R¹³ is a variable related to Y—for example when Y is CH₂CH(CH₂OCH₃) then R¹³ is CH₂OCH₃; LG is a leaving group). (C) is then coupled to the appropriate acid (2) and the acid protecting group R¹² is then removed by well known methods in the art, for example, trifluoroacetic acid or potassium hydroxide.

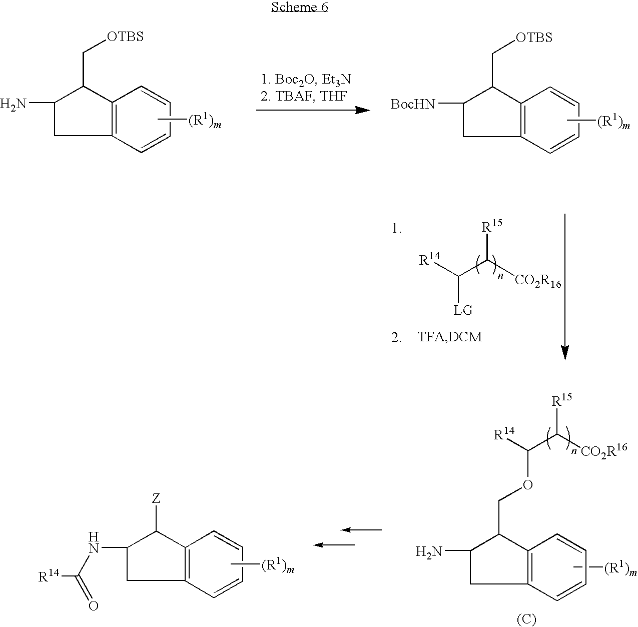

(wherein R¹⁶ is (1-6C)alkyl, R¹⁴ and R¹⁵ are variables related to Y—for example when Y is CH₂OCH(CH₃)CH₂ then R¹⁴ is CH₃ and R¹⁵ is H; LG is a leaving group). (C) is then coupled to the appropriate acid (2) and the acid protecting group R¹⁶ is then removed by known methods in the art, for example, trifluoroacetic acid or potassium hydroxide.

It will be appreciated that certain of the various ring substituents in the compounds of the present invention, for example R¹ and R⁴, may be introduced by standard aromatic substitution reactions or generated by conventional functional group modifications either prior to or immediately following the processes mentioned above, and as such are included in the process aspect of the invention. Such reactions may convert one compound of the formula (1) into another compound of the formula (1). Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Certain intermediates in the preparation of a compound of the formula (1) are novel and form another aspect of the invention.

As stated hereinbefore the compounds defined in the present invention possesses glycogen phosphorylase inhibitory activity. This property may be assessed, for example, using the procedure set out below.

### Assay

The activity of the compounds is determined by measuring the inhibitory effect of the compounds on glycogen degradation, the production of glucose-1-phosphate from glycogen is monitored by the multienzyme coupled assay, as described in EP 0 846 464 A2, general method of Pesce et al (Pesce, M A, Bodourian, S H, Harris, R C, and Nicholson, J F (1977) Clinical Chemistry 23, 1171-1717). The reactions were in 384 well microplate format in a volume of 50 μl. The change in fluorescence due to the conversion of the co-factor NAD to NADH is measured at 340 nM excitation, 465 nm emission in a Tecan Ultra Multifunctional Microplate Reader. The reaction is in 50 mM HEPES, 3.5 mM KH₂PO₄, 2.5 mM MgCl₂, 2.5 mM ethylene glycol-bis(b-aminoethyl ether) N,N,N′N′-tetraacetic acid, 100 mM KCl, 8 mM D-(+)-glucose pH7.2, containing 0.5 mM dithiothreitol, the assay buffer solution. Human recombinant liver glycogen phosphorylase a (hrl GPa) 20 nM is pre-incubated in assay buffer solution with 6.25 mM NAD, 1.25 mg type III glycogen at 1.25 mg ml⁻¹ the reagent buffer, for 30 minutes. The coupling enzymes, phosphoglucomutase and glucose-6-phosphate dehydrogenase (Sigma) are prepared in reagent buffer, final concentration 0.25 Units per well. 20 μl of the hrl GPa solution is added to 10 μl compound solution and the reaction started with the addition of 20 ul coupling enzyme solution. Compounds to be tested are prepared in 10 μl 5% DMSO in assay buffer solution, with final concentration of 1% DMSO in the assay. The non-inhibited activity of GPa is measured in the presence of 10 μl 15% DMSO in assay buffer solution and maximum inhibition measured in the presence of 5 mgs ml⁻¹ N-ethylmaleimide. After 6 hours at 30° C. Relative Fluoresence Units (RFUs) are measured at 340 nM excitation, 465 nm emission.

The assay is performed at a test concentration of inhibitor of 10 μM or 100 μM. Compounds demonstrating significant inhibition at one or both of these concentrations may be further evaluated using a range of test concentrations of inhibitor to determine an IC₅₀, a concentration predicted to inhibit the enzyme reaction by 50%.

Activity is calculated as follows:—

% inhibition=(1−(compound RFUs−fully inhibited RFUs)/(non-inhibited rate RFUs−fully inhibited RFUs))*100.

Typical IC₅₀ values for compounds of the invention when tested in the above assay are in the range 100 μM to 1 nM. For example, Example 5 was found to have an IC₅₀ of 0.161 μM, and Example 2 was found to have an IC₅₀ of 1.67 μM.

The inhibitory activity of compounds was further tested in rat primary hepatocytes. Rat hepatocytes were isolated by the collagenase perfusion technique, general method of Seglen (P. O, Seglen, Methods Cell Biology (1976) 13 29-83). Cells were cultured on Nunclon six well culture plates in DMEM (Dulbeco's Modified Eagle's Medium) with high level of glucose containing 10% foetal calf serum, NEAA (non essential amino acids), Glutamine, penicillin/streptomycin ((100 units/100 ug)/ml) for 4 to 6 hours. The hepatocytes were then cultured in the DMEM solution without foetal calf serum and with 10 nM insulin and 10 nM dexamethasone. Experiments were initiated after 18-20 hours culture by washing the cells and adding Krebs-Henseleit bicarbonate buffer containing 2.5 mM CaCl₂ and 1% gelatin. The test compound was added and 5 minutes later the cells were challenged with 25 nM glucagon. The Krebs-Henseleit solution was removed after 60 min incubation at 37° C., 95%O₂/5% CO₂ and the glucose concentration of the Krebs-Henseleit solution measured.

According to a further aspect of the invention there is provided a pharmaceutical composition which comprises a compound of the formula (1), or a pharmaceutically acceptable salt or in vivo hydrolysable ester thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents. In one aspect, the compositions of the invention are in a form suitable for oral dosage.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The compound of formula (1) will normally be administered to a warm-blooded animal at a unit dose within the range 5-5000 mg per square meter body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

The inhibition of glycogen phosphorylase activity described herein may be applied as a sole therapy or may involve, in addition to the subject of the present invention, one or more other substances and/or treatments. Such conjoint treatment may be achieved by way of the simultaneous, sequential or separate administration of the individual components of the treatment. Simultaneous treatment may be in a single tablet or in separate tablets.

For example, in order to prevent, delay or treat type 2 diabetes mellitus, the compounds of the present invention or their pharmaceutically acceptable salts may be administered in combination with one or more of the following agent(s):
1) Insulin and insulin analogues;
2) Insulin secretagogues including sulphonylureas (for example glibenclamide, glipizide), prandial glucose regulators (for example repaglinide, nateglinide) and glucokinase activators
3) Agents that improve incretin action (for example dipeptidyl peptidase IV inhibitors, GLP-1 agonists)
4) Insulin sensitising agents including PPARgamma agonists (for example pioglitazone and rosiglitazone); and agents with combined PPARalpha and gamma activity
5) Agents that modulate hepatic glucose balance (for example metformin, fructose 1, 6 bisphosphatase inhibitors, glycogen synthase kinase inhibitors, glucokinase activators)
6) Agents designed to reduce the absorption of glucose from the intestine (for example acarbose);
7) Agents that prevent the reabsorption of glucose by the kidney (SGLT inhibitors)
8) Agents designed to treat the complications of prolonged hyperglycaemia (for example aldose reductase inhibitors)
9) Anti-obesity agents (for example sibutramine and orlistat);
10) Anti-dyslipidaemia agents such as, HMG-CoA reductase inhibitors (statins, e.g. pravastatin); PPARα agonists (fibrates, e.g. gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations);
11) Antihypertensive agents such as, β blockers (e.g. atenolol, inderal); ACE inhibitors (e.g. lisinopril); Calcium antagonists (e.g. nifedipine); Angiotensin receptor antagonists (e.g. candesartan), α antagonists and diuretic agents (e.g. furosemide, benzthiazide);
12) Haemostasis modulators such as, antithrombotics, activators of fibrinolysis and antiplatelet agents; thrombin antagonists; factor Xa inhibitors; factor VIIa inhibitors); antiplatelet agents (e.g. aspirin, clopidogrel); anticoagulants (heparin and Low molecular weight analogues, hirudin) and warfarin;
13) Agents which antagonise the actions of glucagon; and
14) Anti-inflammatory agents, such as non-steroidal anti-inflammatory drugs (e.g. aspirin) and steroidal anti-inflammatory agents (e.g. cortisone).

According to a further aspect of the present invention there is provided a compound of the formula (1), or a pharmaceutically acceptable salt or in vivo hydrolysable ester thereof, as defined hereinbefore, for use in a method of treatment of a warm-blooded animal such as man by therapy.

According to an additional aspect of the invention there is provided a compound of the formula (1), or a pharmaceutically acceptable salt or in vivo hydrolysable ester thereof, as defined hereinbefore, for use as a medicament.

According to an additional aspect of the invention there is provided a compound of the formula (1), or a pharmaceutically acceptable salt or in vivo hydrolysable ester thereof, as defined hereinbefore, for use as a medicament in the treatment of type 2 diabetes, insulin resistance, syndrome X, hyperinsulinaemia, hyperglucagonaemia, cardiac ischaemia or obesity in a warm-blooded animal such as man.

According to this another aspect of the invention there is provided the use of a compound of the formula (1), or a pharmaceutically acceptable salt or in vivo hydrolysable ester thereof, as defined hereinbefore in the manufacture of a medicament for use in the treatment of type 2 diabetes, insulin resistance, syndrome X, hyperinsulinaemia, hyperglucagonaemia, cardiac ischaemia or obesity in a warm-blooded animal such as man.

According to this another aspect of the invention there is provided the use of a compound of the formula (1), or a pharmaceutically acceptable salt or in vivo hydrolysable ester thereof, as defined hereinbefore in the manufacture of a medicament for use in the treatment of type 2 diabetes in a warm-blooded animal such as man.

According to a further feature of this aspect of the invention there is provided a method of producing a glycogen phosphorylase inhibitory effect in a warm-blooded animal, such as man, in need of such treatment which comprises administering to said animal an effective amount of a compound of formula (1).

According to this further feature of this aspect of the invention there is provided a method of treating type 2 diabetes, insulin resistance, syndrome X, hyperinsulinaemia, hyperglucagonaemia, cardiac ischaemia or obesity in a warm-blooded animal, such as man, in need of such treatment which comprises administering to said animal an effective amount of a compound of formula (1).

According to this further feature of this aspect of the invention there is provided a method of treating type 2 diabetes in a warm-blooded animal, such as man, in need of such treatment which comprises administering to said animal an effective amount of a compound of formula (1).

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

In addition to their use in therapeutic medicine, the compounds of formula (1) and their pharmaceutically acceptable salts are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following examples in which, unless stated otherwise:

(i) temperatures are given in degrees Celsius (° C.); operations were carried out at room or ambient temperature, that is, at a temperature in the range of 18-25° C. and under an atmosphere of an inert gas such as argon;
(ii) organic solutions were dried over anhydrous magnesium sulphate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates; where a Bond Elut column is referred to, this means a column containing 10 g or 20 g or 50 g of silica of 40 micron particle size, the silica being contained in a 60 ml disposable syringe and supported by a porous disc, obtained from Varian, Harbor City, Calif., USA under the name “Mega Bond Elut SI”; “Mega Bond Elut” is a trademark; where a Biotage cartridge is referred to this means a cartridge containing KP-SIL™ silica, 60μ, particle size 32-63 mM, supplied by Biotage, a division of Dyax Corp., 1500 Avon Street Extended, Charlottesville, Va. 22902, USA;
(iv) in general, the course of reactions was followed by TLC and reaction times are given for illustration only;
(v) yields are given for illustration only and are not necessarily those which can be obtained by diligent process development; preparations were repeated if more material was required;
(vi) where given, NMR data is in the form of delta values for major diagnostic protons, given in parts per million (ppm) relative to tetramethylsilane (TMS) as an internal standard, determined at 300 MHz using perdeuterio dimethyl sulphoxide (DMSO-δ₆) as solvent unless otherwise indicated, other solvents (where indicated in the text) include deuterated chloroform CDCl₃;
(vii) chemical symbols have their usual meanings; SI units and symbols are used;
(viii) reduced pressures are given as absolute pressures in Pascals (Pa); elevated pressures are given as gauge pressures in bars;
(ix) solvent ratios are given in volume:volume (v/v) terms;
(x) Mass spectra (MS) data was generated on an LCMS system where the HPLC component comprised generally either a Waters Alliance HT (2790 & 2795) equipment and was run on a Phemonenex Gemini C18 5 μm, 50×2 mm column (or similar) eluting with either acidic eluent (for example, using a gradient between 0-95% water/acetonitrile with 5% of a 1% formic acid in 50:50 water:acetonitrile (v/v) mixture; or using an equivalent solvent system with methanol instead of acetonitrile), or basic eluent (for example, using a gradient between 0-95% water/acetonitrile with 5% of a 0.1% 880 Ammonia in acetonitrile mixture); and the MS component comprised generally a Waters ZQ spectrometer. Chromatograms for Electrospray (ESI) positive and negative Base Peak Intensity, and UV Total Absorption Chromatogram from 220-300 nm, are generated and values for m/z are given; generally, only ions which indicate the parent mass are reported and unless otherwise stated the value quoted is (MH⁺);
(xi) The following abbreviations may be used:
RT retention time
EtOAc ethyl acetate;
MeOH methanol;
EtOH ethanol;
DCM dichloromethane;
HOBT 1-hydroxybenzotriazole;
DIPEA di-isopropylethylamine;
EDCI 1-ethyl-3-(3-dimethylaminopropyl)carbodi-imide hydrochloride;
Et₂O diethyl ether;
THF tetrahydrofuran;
DMF N,N-dimethylformamide;
HATU O-(7-Azabenzotriazol-1-yl)-N,N,N′,N′-tetramethyluroniumhexafluorophosphate
EDAC 1-(3-dimethylaminopropyl)-3-ethyl-carbodiimide hydrochloride
TFA Trifluoroacetic acid
DMTMM 4-(4,6-Dimethoxy-1,3,5-triazin-2-yl)-4-methylmorpholinium chloride
DMA N,N-dimethylacetamide
NaHCO₃ Sodium bicarbonate
NaHMDS Sodium hexamethyldisilazide mCPBA meta-chloroperbenzoic acid
DABCO diaza-[2.2.2]bicyclo-octane
AcOH acetic acid
TEA triethylamine
Boc tert-butoxycarbonate
MeCN acetonitrile

### Example 1

### [((1R,2R)-2-{[(5-Chloro-1H-indol-2-yl)carbonyl]amino}-2,3-dihydro-1H-inden-1-yl)methoxy]acetic acid

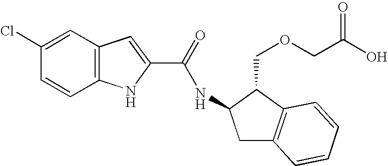

To a solution of tert-butyl[((1R,2R)-2-{[(5-chloro-1H-indol-2-yl)carbonyl]amino}-2,3-dihydro-1H-inden-1-yl)methoxy]acetate (Intermediate 1; 0.5 g, 1.1 mmol) in DCM (5 mL) was added trifluoroacetic acid (1 mL) and the reaction stirred at ambient temperature for 2 h. Evaporation under reduced pressure and drying in vacuo gave the title compound (275 mg, 63%) as a foam.

¹H NMR δ: 2.9 (dd, 1H), 3.25 (dd, 1H), 3.47 (m, 1H), 3.7 (m, 1H), 3.82 (m, 1H), 4.05 (s, 2H), 4.55 (m, 1H), 7.2 (m, 6H), 7.4 (m, 2H), 7.7 (s, 1H), 8.75 (d, 1H), 11.74 (s, 1H) MS m/z 397/399.

The following example was made by the process of Example 1 using the appropriate tert-butyl ester (Intermediate 2) as starting material.

### Example 2

### [((1R,2R)-2-{[(5-Fluoro-1H-indol-2-yl)carbonyl]amino}-2,3-dihydro-1H-inden-1-yl)methoxy]acetic acid

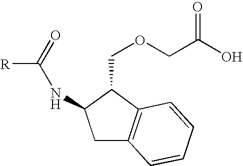

| Example | R | ¹H NMR | M/z |
|---|---|---|---|
| 2 | 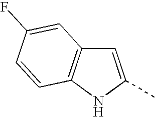 | 2.9 (dd, 1H), 3.27 (dd, 1H), 3.45 (m, 1H), 3.7 (m, 1H), 3.82 (m, 1H), 4.05 (s, 2H), 4.57 (m, 1H), 7.02 (ddd, 1H), 7.17 (m, 5H), 7.39 (m, 3H), 8.7 (d, 1H), 11.63 (s, 1H) | 381/382 |

### Example 3

### (2R/S)-[((1R,2R)-2-{[(5-chloro-1H-indol-2-yl)carbonyl]amino}-2,3-dihydro-1H-inden-1-yl)methoxy]propanoic acid

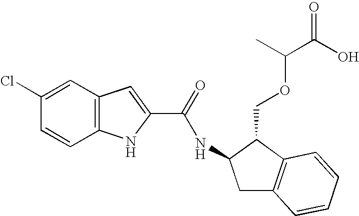

To a solution of tert-butyl (2R/S)-[((1R,2R)-2-{[(5-chloro-1H-indol-2-yl)carbonyl]amino}-2,3-dihydro-1H-inden-1-yl)methoxy]propanoate (Intermediate 13; 430 mg, 0.917 mmol) in DCM (10 mL) was added trifluoroacetic acid (1 mL) and the reaction stirred at ambient temperature for 20 h. Evaporation under reduced pressure and drying in vacuo gave the title compound (340 mg, 90%) as a powder.

¹H NMR δ: 1.27 (dd, 3H), 2.9 (m, 1H), 3.25 (m, 1H), 3.45 (m, 1.5H), 3.63 (m, 0.5H), 3.8 (m, 0.5H), 3.95 (m, 1.5H), 4.55 (m, 1H), 7.2 (m, 6H), 7.4 (m, 2H), 7.68 (d, 1H), 8.75 (d, 1H), 11.74 (s, 0.5H), 12.52 (s, 0.5H); MS m/z 411/413 (M−H)

The following example was made by the process of EXAMPLE 3 using the appropriate tert-butyl ester (Intermediate 14) as starting material:

### Example 4

### (2R/S)-[((1R,2R)-2-{[(5-fluoro-1H-indol-2-yl)carbonyl]amino}-2,3-dihydro-1H-inden-1-yl)methoxy]propanoic acid

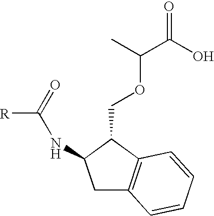

| Example | R | ¹H NMR (CDCl₃) | M/z |
|---|---|---|---|
| 4 | 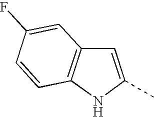 | 1.4 (dd, 3H), 2.95 (m, 1H), 3.5 (m, 2.5H), 3.77 (dd, 0.5H), 3.95 (dd, 0.5H), 4.05 (m, 1.5H), 4.9 (m, 1H), 6.68 (d, 1H), 6.82 (dd, 1H), 7.02 (ddd, 1H), 7.2 (m, 6H), 7.35 (dd, 1H), 9.75 (s, 1H) | 395 (M − H) |

### Example 5

### ((1R,2R)-2-{[(5-Fluoro-1H-indol-2-yl)carbonyl]amino}-2,3-dihydro-1H-inden-1-yl)acetic acid

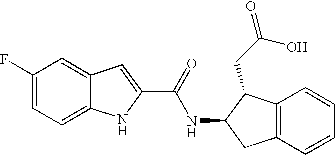

Methyl ((1R,2R)-2-{[(5-fluoro-1H-indol-2-yl)carbonyl]amino}-2,3-dihydro-1H-inden-1-yl)acetate (Intermediate 18; 100 mg, 0.27 mmol) was dissolved in MeOH (5 mL). Potassium carbonate (500 mg) was added and the suspension stirred at 60° C. for 19 h. The volatiles were removed under reduced pressure then EtOAc (25 mL) and water (25 mL) were added. The organic phase was separated, washed with water (2×25 mL), brine (25 mL), dried (MgSO₄) and the volatiles removed under reduced pressure. The product was then dried in vacuo to afford the title compound (34 mg, 36%) as a solid.

¹H-NMR δ: 2.59 (dd, 1H), 2.69 (dd, 1H), 2.91 (dd, 1H), 3.25 (dd, 1H), 3.61 (m, 1H), 4.46 (m, 1H), 7.01 (t, 1H), 7.19 (m, 5H), 7.39 (m, 2H), 8.68 (d, 1H), 11.64 (s, 1H), 12.22 (s, 1H); MS m/z 353.

The following example was made by the process of Example 5 using indol-2-yl)carbonyl]amino}-2,3-dihydro-1H-inden-1-yl)acetate (Intermediate 19) as starting material:

### Example 6

### ((1R,2R)-2-{[(5-Chloro-1H-indol-2-yl)carbonyl]amino}-2,3-dihydro-1H-inden-1-yl)acetic acid

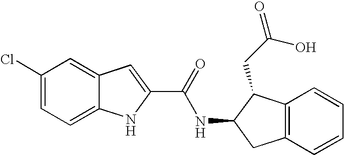

¹H-NMR δ: 2.63 (m, 2H), 2.91 (dd, 1H), 3.25 (dd, 1H), 3.61 (m, 1H), 4.46 (m, 1H), 7.19 (m, 6H), 7.42 (d, 1H), 7.68 (s, 1H), 8.72 (d, 1H), 11.73 (s, 1H), 12.20 (s, 1H); MS m/z 369.

### Example 7

### ((1R,2R)-2-{[(5,6-difluoro-1H-indol-2-yl)carbonyl]amino}-2,3-dihydro-1H-inden-1-yl)acetic acid

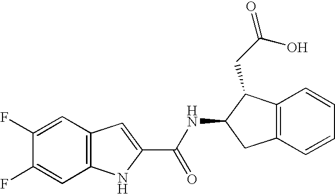

Methyl ((1R,2R)-2-{[(5,6-difluoro-1H-indol-2-yl)carbonyl]amino}-2,3-dihydro-1H-inden-1-yl)acetate (Intermediate 25, 132 mg, 0.34 mmol) was suspended in a mixture of methanol (5 mL) and 2M sodium hydroxide (1.71 mL, 3.42 mmol) and heated to 50° C. for 30 min, giving a clear solution. The reaction mixture was then concentrated under reduced pressure and acidified to pH2 with concentrated HCl. The resulting precipitate was extracted into ethyl acetate (50 mL) and the ethyl acetate solution washed with water (20 mL) and brine (20 mL), dried (MgSO₄) and evaporated to give the title compound as a white solid. (115 mg, 91%).

¹H NMR δ: 2.6 (m, 2H), 2.9 (dd, 1H), 3.2 (m, 1H), 3.6 (m, 1H), 4.45 (m, 1H), 7.2 (m, 5H), 7.3 (dd, 1H), 7.6 (dd, 1H), 8.7 (d, 1H), 11.7 (s, 1H), 12.2 (s, 1H); MS m/z 383, 385.

### Example 8

### {(1R,2R)-2-[(5-Chloro-7-fluoro-1H-indole-2-carbonyl)-amino]-indan-1-yl}-acetic acid

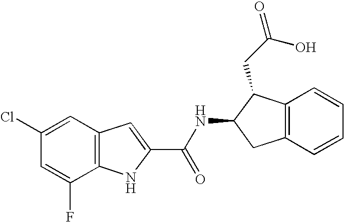

{(1R,2R)-2-[(5-Chloro-7-fluoro-1H-indole-2-carbonyl)-amino]-indan-1-yl}-acetic acid methyl ester (Intermediate 28, 202 mg, 0.51 mmol) was suspended in methanol (10 mL) and treated at ambient temperature with a 2M solution of sodium hydroxide (2.5 mL). The mixture was heated to 50° C. for 2 h and then cooled to ambient and concentrated under reduced pressure. The residue was dissolved in water (20 mL), acidified to pH3 with 2M HCl and extracted with ethyl acetate (3×10 mL). The combined extracts were washed with water (2×10 mL), dried (MgSO₄) and evaporated to leave the title compound as a white solid. (150 mg, 76%).

¹H NMR (400 MHz, DMSO-d₆) δ 2.58-2.74 (2H, m), 2.89-2.95 (1H, m), 3.21-3.34 (1H, m), 3.59-3.64 (1H, m), 4.45-4.52 (1H, m), 7.15-7.26 (6H, m), 7.58-7.59 (1H, s), 8.76 (1H, d), 12.25 (2H, s); MS m/z 387

### Examples 9 and 10

### (2R)-2-{(1R,2R)-2-[(5-Chloro-1H-indole-2-carbonyl)-amino]-indan-1-yl}-4-methoxy-butyric acid and (2S)-2-{(1R,2R)-2-[(5-Chloro-1H-indole-2-carbonyl)-amino]-indan-1-yl}-4-methoxy-butyric acid

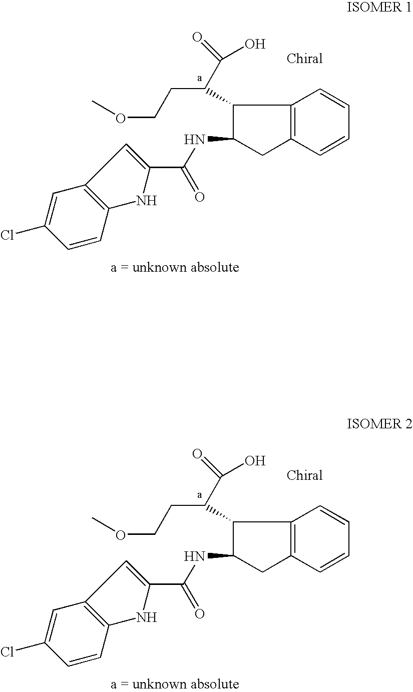

Dimethyl ((1S,2R)-2-{[(2,3-dichloro-4H-thieno[3,2-b]pyrrol-5-yl)carbonyl]amino}-2,3-dihydro-1H-inden-1-yl)(2-methoxyethyl)malonate (Intermediate 31, 200 mg, 0.44 mmol) was dissolved in THF (10 mL) before adding lithium hydroxide (50 mg, 1.2 mmol) and water (3 mL). The reaction was heated at 130° C. by microwaves for 30 mins before adding EtOAc (25 mL), it was washed with 1N citric acid (10 mL), water (10 ml), brine (10 mL), dried (MgSO₄) and the volatiles removed under reduced pressure to give a brown oil. This oil was purified by HPLC (acetonitrile/water, TFA buffer 0.2%) to give the product (64 mg, 0.14 mmol, 32%). The product was then chromatographed under the following conditions to separate the two diastereoisomers:

| | |
|---|---|
| Column | Merck 50 mm 16 μm Kromasil Chirose 2, |
| | CT9014 Packed 16-02-04 |
| Eluent | iso-Hexane/EtOAc/AcOH/TEA 50/50/0.2/0.1 |

The appropriate fractions were combined and evaporated before dissolving each diastereoisomer in EtOAc (50 mL) and acidifying with TFA (2 mL) then washing with water (2×25 mL). The products were then dried in vacuo to afford a first eluting compound (20 mg, 0.047 mmol, 33%) and a second eluting compound (27 mg, 0.063 mmol, 45%) as gums, one of which is (2R)-2-{(1R,2R)-2-[(5-chloro-1H-indole-2-carbonyl)-amino]-indan-1-yl}-4-methoxy-butyric acid and the other of which is (2S)-2-{(1R,2R)-2-[(5-chloro-1H-indole-2-carbonyl)-amino]-indan-1-yl}-4-methoxy-butyric acid: First eluting (Example 9): ¹H NMR (400 MHz, CDCl₃) 1.09-1.21 (1H, m), 1.91 (1H, s), 3.04-3.06 (1H, m), 3.13 (3H, s), 3.35 (2H, t), 3.56 (1H, t), 3.81-3.84 (1H, m), 4.45 (1H, m), 6.89 (1H, s), 7.10 (3H, t), 7.08-7.15 (2H, m), 7.20 (1H, s), 7.29 (1H, d), 7.44 (1H, d); MS m/z 449[M+Na].

Second eluting (Example 10): ¹H NMR (400 MHz, CDCl₃) δ1.24 (1H, d), 3.18 (2H, s), 3.36 (2H, q), 3.42 (1H, s), 4.65 (1H, m), 6.83 (1H, s), 7.07-7.09 (2H, m), 7.10 (1H, s), 7.21 (1H, m), 7.46 (1H, s); MS m/z 449[M+Na].

### Examples 11 and 12

### (2R)-2-{(1R,2R)-2-[(5-Chloro-1H-indole-2-carbonyl)-amino]-indan-1-yl}-propionic acid and (2S)-2-{(1R,2R)-2-[(5-Chloro-1H-indole-2-carbonyl)-amino]-indan-1-yl}-propionic acid

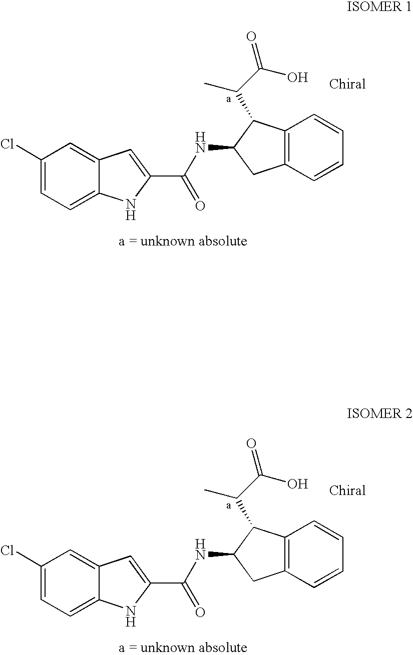

2-{(1R,2R)-2-[(5-Chloro-1H-indole-2-carbonyl)-amino]-indan-1-yl}-propionic acid methyl ester (Intermediate 36; 120 mg, 0.3 mmol) was dissolved in THF (10 mL) before adding a water (3 mL) solution of lithium hydroxide (25 mg, 0.60 mmol). The solution was stirred at room temperature for 48 h. The volatiles were removed under reduced pressure then EtOAc (25 mL) and a 1N solution of citric acid (10 mL) were added. The organic phase was separated, washed with water (2×10 mL), brine (10 mL), dried (MgSO₄) and the volatiles removed under reduced pressure to give an oil. This oil was purified by HPLC (acetonitrile/water, TFA buffer 0.2%) to give the product (56 mg, 0.15 mmol, 50%)

The product (56 mg, 0.15 mmol) was chromatographed under the following conditions to separate the two diastereoisomers:

| | |
|---|---|
| Column | 10 μm Merck 50 mm Kromasil 100 CHI-TBB No. CT0021 |
| Eluent | iso-Hexane/EtOH/HOAc/TEA 80/20/0.2/0.1 |

The appropriate fractions were combined and evaporated before dissolving each diastereoisomer in EtOAc (10 mL) and acidifying with TFA (1 mL) then washing with water (2×10 mL). The products were then dried in vacuo to afford a first eluting compound (27.0 mg, 0.07 mmol, 46%) and a second eluting compound (19.8 mg, 0.05 mmol, 33%) as gums, one of which is (2R)-2-{(1R,2R)-2-[(5-chloro-1H-indole-2-carbonyl)-amino]-indan-1-yl}-propionic acid and one of which is (2S)-2-{(1R,2R)-2-[(5-chloro-1H-indole-2-carbonyl)-amino]-indan-1-yl}-propionic acid:

First eluting (Example 11): ¹H NMR (400 MHz, CDCl₃) δ 2.15 (1H, s), 2.77 (2H, m), 3.14 (1H, s), 3.63 (1H, d), 3.88 (1H, s), 4.38 (1H, s), 6.88 (1H, s), 7.12 (4H, s), 7.19 (3H, s), 7.29 (1H, d), 7.37-7.42 (1H, m), 8.97 (1H, s), 10.10 (1H, s); MS m/z 405[M+Na].

Second eluting (Example 12): ¹H NMR (400 MHz, CDCl₃) δ 2.15 (1H, s), 2.63 (1H, s), 2.70-2.75 (1H, m), 3.41-3.44 (1H, m), 3.61 (1H, s), 4.62 (1H, s), 6.80 (1H, s), 7.12 (5H, s), 7.19 (3H, s), 7.25 (2H, s), 7.46 (1H, s), 10.10 (1H, s); MS m/z 405[M+Na].

### Intermediate 1: tert-butyl[((1R,2R)-2-{[(5-chloro-1H-indol-2-yl)carbonyl]amino}-2,3-dihydro-1H-inden-1-yl)methoxy]acetate

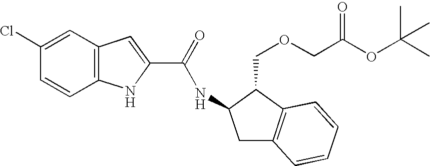

HOBT (280 mg, 2.07 mmol), tert-butyl {[(1R,2R)-2-amino-2,3-dihydro-1H-inden-1-yl]methoxy}acetate (Intermediate 3; 575 mg, 2.07 mmol) and EDAC (496 mg, 2.6 mmol) were added to a suspension of 5-chloroindole-2-carboxylic acid (404 mg, 2.07 mmol) in DMA (5 mL). The reaction was stirred at ambient temperature for 20 h. Water (25 mL) was added and the precipitate filtered, washed with water (2×20 mL) and dried. Purification by flash chromatography (SiO₂, iso-hexane:EtOAc, 1:1) gave the title compound (500 mg, 53%) as a foam.

¹H NMR δ: 1.48 (s, 9H), 3.0 (dd, 1H), 3.55 (m, 1H), 3.65 (dd, 1H), 3.8 (m, 1H), 3.95 (m, 1H), 4.05 (d, 2H), 4.61 (m, 1H), 6.86 (s, 1H), 7.05 (m, 1H), 7.47 (m, 5H), 7.35 (d, 1H), 7.59 (s, 1H), 9.63 (s, 1H); MS m/z 453/455 (M−H)

The following intermediate was made by the process of Intermediate 1, using tert-butyl {[(1R,2R)-2-amino-2,3-dihydro-1H-inden-1-yl]methoxy}acetate (Intermediate 3) as the amine and the appropriate carboxylic acid (5-fluoroindole-2-carboxylic acid)

### Intermediate 2: tert-butyl[((1R,2R)-2-{[(5-fluoro-1H-indol-2-yl)carbonyl]amino}-2,3-dihydro-1H-inden-1-yl)methoxy]acetate

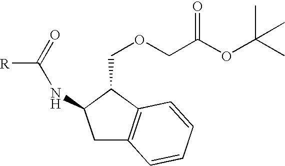

| Intermediate | R | ¹H NMR (CDCl₃) | M/z |
|---|---|---|---|
| 2 | 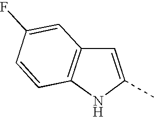 | 1.48 (s, 9H), 2.98 (dd, 1H), 3.55 (m, 1H), 3.64 (dd, 1H), 3.8 (m, 1H), 3.95 (m, 1H), 4.05 (d, 2H), 4.62 (m, 1H), 6.9 (s, 1H), 7.02 (ddd, 1H), 7.03 (m, 1H), 7.24 (m, 5H), 7.35 (dd, 1H), 9.56 (s, 1H) | 437 (M − H) |

### Intermediate 3: tert-Butyl{[(1R,2R)-2-amino-2,3-dihydro-1H-inden-1-yl]methoxy}acetate

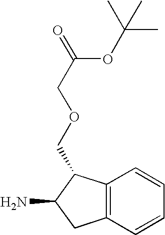

To a solution of tert-butyl ({(1R,2R)-2-[({[tert-butyl(dimethyl)silyl]oxy}carbonyl)amino]-2,3-dihydro-1H-inden-1-yl}methoxy)acetate (Intermediate 4; 3.5 g, 8.03 mmol) in THF (30 mL) was added tetra-n-butyl ammoniumfluoride (8.8 mL, 1M in THF, 8.8 mmol) and the reaction stirred at ambient temperature for 1 h. ammonium chloride solution (25 mL, saturated aqueous) was added and the mixture extracted with EtOAc (2×25 mL). The organic extracts were washed with water (20 mL), brine (20 mL), dried (MgSO₄) and the volatiles removed by evaporation under reduced pressure to give the title compound (2.2 g, 100%) as an oil.

MS m/z 278.

### Intermediate 4: tert-Butyl ({(1R,2R)-2-[({[tert-butyl(dimethyl)silyl]oxy}carbonyl)amino]-2,3-dihydro-1H-inden-1-yl}methoxy)acetate

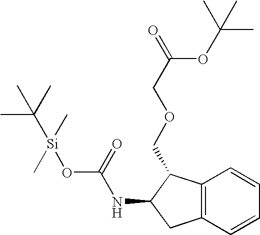

To a solution of tert-butyl ({(1R,2R)-2-[(tert-butoxycarbonyl)amino]-2,3-dihydro-1H-inden-1-yl}methoxy)acetate (Intermediate 5; 2.8 g, 7.42 mmol) and 2,6-lutidine (1.73 mL, 14.83 mmol) in anhydrous DCM (20 mL) was added tert-butyl dimethyl silyl trifluoromethanesulphonate (2.6 mL, 11.1 mmol) and the reaction stirred at ambient temperature for 30 mins. Ammonium chloride solution (20 mL, saturated aqueous) was added and the mixture extracted with EtOAc (2×3 5 mL). The organic extracts were washed with water (20 mL), brine (20 mL), dried (MgSO₄) and the volatiles removed by evaporation under reduced pressure to give the title compound (3.2 g, 100%) as an oil. MS m/z 458 (M+Na).

### Intermediate 5: tert-Butyl ([(1R,2R)-2-[(tert-butoxycarbonyl)amino]-2,3-dihydro-1H-inden-1-yl]methoxy)acetate

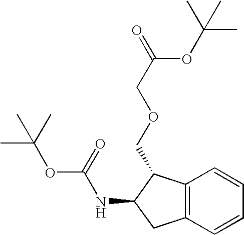

To a solution of tert-butyl[(1R,2R)-1-(hydroxymethyl)-2,3-dihydro-1H-inden-2-yl]carbamate (Intermediate 6; 2.63 g, 10.0 mmol) in DCM (35 mL) was added tert-butylbromo acetate (2.0 mL, 12.5 mmol), tetra-n-butylammonium hydrogen sulphate (850 mg, 2.5 mmol) and sodium hydroxide (9.6 mL, 50% w/v aqueous, 120.0 mmol) and the reaction stirred at ambient temperature for 3 h. Water (50 mL) was added and the mixture extracted with DCM (2×50 mL). The organic extracts were washed with water (25 mL), brine (25 mL), dried (MgSO₄) and the volatiles removed under reduced pressure. The residue was purified by flash chromatography with (SiO₂, iso-hexane:EtOAc, 3:1) gave the title compound (350 mg, 93%) as an oil. MS m/z 400 (M+Na).

### Intermediate 6: tert-Butyl[(1R,2R)-1-(hydroxymethyl)-2,3-dihydro-1H-inden-2-yl]carbamate

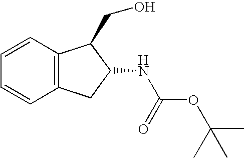

Tetrabutylammonium fluoride (10.0 mL, 2.0M in THF, 20.0 mmol) was added to a solution of tert-butyl[(1R,2R)-1-({[tert-butyl(dimethyl)silyl]oxy}methyl)-2,3-dihydro-1H-inden-2-yl]carbamate (Intermediate 7; 4.1 g, 10.9 mmol) in THF (50 mL) and stirred at ambient temperature for 4 h. The volatiles were removed under reduced pressure and the residue dissolved in ethyl acetate (100 mL), washed with water (2×50 mL), brine (50 mL), dried (MgSO₄) and the volatiles removed under reduced pressure. The crude residue was triturated (4:1, iso-hexane:ethyl acetate), filtered and dried to give the title compound (1.5 g, 54%) as white solid.

¹H NMR 1.44 (s, 9H), 2.78 (dd, 1H), 3.15 (m, 2H), 3.61 (m, 1H), 3.75 (m, 1H), 4.07 (m, 1H), 4.7 (m, 1H), 7.19 (m, 4H), 7.37 (m, 1H).

### Intermediate 7: tert-Butyl[(1R,2R)-1-({[tert-butyl(dimethyl)silyl]oxy}methyl)-2,3-dihydro-1H-inden-2-yl]carbamate

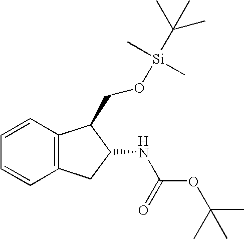

(1R,2R)-1-({[tert-Butyl(dimethyl)silyl]oxy}methyl)-2,3-dihydro-1H-inden-2-yl]amine (Intermediate 8; 3.1 g, 11.2 mmol) and triethylamine (3.1 mL, 22.4 mmol) were dissolved in DCM (40 μL). Di-tert-butyl dicarbonate (2.9 g, 13.4 mmol) in DCM (10 mL) was added and the mixture stirred at ambient temperature for 24 h. The volatiles were removed under reduced pressure and the residue dissolved in ethyl acetate (75 mL), washed with water (2×50 mL), brine (50 mL), dried (MgSO₄) and the volatiles removed under reduced pressure. The crude residue was purified by silica gel chromatography (16:1, iso-hexane:ethyl acetate) to give the title compound (4.2 g, 100%) as a colourless oil.

¹H NMR 0.3 (d, 6H), 0.85 (s, 9H), 1.42 (s, 9H), 2.75 (dd, 1H), 3.15 (m, 2H), 3.79 (m, 1H), 3.95 (m, 1H), 4.05 (m, 1H), 7.15 (m, 4H), 7.3 (m, 1H).

### Intermediate 8: [(1R,2R)-1-({[tert-Butyl(dimethyl)silyl]oxy}methyl)-2,3-dihydro-1H-inden-2-yl]amine

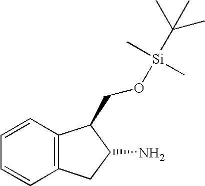

(1S,2S)-1-({[tert-Butyl(dimethyl)silyl]oxy}methyl)-2,3-dihydro-1H-inden-2-yl methanesulfonate (Intermediate 9; 7.2 g, 20.2 mmol) was dissolved in DMA (50 mL), sodium azide (3.94 g, 60.6 mmol) was added and the mixture stirred at 60° C. for 7 h. The mixture was poured into ethyl acetate (250 mL), washed with water (6×75 mL), brine (100 mL) and dried (MgSO₄). Palladium on carbon (500 mg, 10% w/w) was added, the mixture stirred under a hydrogen atmosphere for 6 h, filtered through Celite and the volatiles removed under reduced pressure to give the title compound (5.2 g, 93%) as a pale brown oil.

¹H NMR 0.07 (d, 6H), 0.9 (s, 9H), 2.58 (dd, 1H), 2.89 (m, 1H), 3.1 (dd, 1H), 3.3 (broad s, 2H), 3.41 (m, 1H), 3.85 (m, 2H), 7.2 (m, 4H).

### Intermediate 9: (1S,2S)-1-({[tert-Butyl(dimethyl)silyl]oxy}methyl)-2,3-dihydro-1H-inden-2-yl methanesulfonate

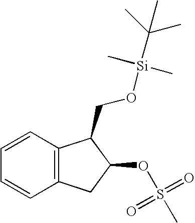

(1S,2S)-1-({[tert-Butyl(dimethyl)silyl]oxy}methyl)indan-2-ol (Intermediate 10; 6.3 g, 22.65 mmol) and triethylamine (4.7 mL, 34.0 mmol) were dissolved in DCM (90 mL) at 5° C. Methanesulfonyl chloride (2.86 g, 24.9 mmol) in DCM (10 mL) was added and the mixture stirred at ambient temperature for 2 h. The volatiles were removed under reduced pressure and the residue dissolved in ethyl acetate (150 mL), washed with water (2×50 mL), brine (50 mL), dried (MgSO₄) and the volatiles removed under reduced pressure. The crude residue was purified by silica gel chromatography (6:1, iso-hexane:ethyl acetate) to give the title compound (7.2 g, 89%) as a colourless oil.

¹H NMR 0.03 (d, 6H), 0.85 (s, 9H), 3.19 (s, 3H), 3.21 (m, 2H), 3.45 (m, 1H), 3.95 (m, 2H), 5.45 (m, 1H), 7.22 (m, 4H).

### Intermediate 10: (1S,2S)-1-({[tert-Butyl(dimethyl)silyl]oxy}methyl)indan-2-ol

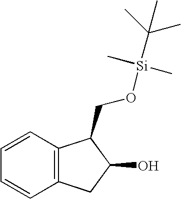

(1S,2S)-1-(Hydroxymethyl)indan-2-ol (Intermediate 11; 9.0 g, 54.8 mmol) and imidazole (4.5 g, 65.8 mmol) were dissolved in DCM (75 mL) at 10° C. tert-Butyldimethylchlorosilane (9.1 g, 60.3 mmol) in DCM (25 mL) was added, the mixture allowed to warm to ambient temperature and stirred for 2 h. The volatiles were removed under reduced pressure and the residue dissolved in ethyl acetate (150 mL), washed with water (2×50 mL), brine (50 mL), dried (MgSO₄) and the volatiles removed under reduced pressure. The crude residue was purified by silica gel chromatography (16:1, iso-hexane:ethyl acetate) to give the title compound (9.5 g, 62%) as a colourless oil.

¹H NMR 0.03 (d, 6H), 0.9 (s, 9H), 2.78 (dd, 1H), 3.0 (dd, 1H), 3.1 (m, 1H), 3.9 (m, 2H), 4.54 (m, 1H), 4.68 (d, 1H), 7.2 (m, 4H).

### Intermediate 11: (1S,2S)-1-(Hydroxymethyl)indan-2-ol

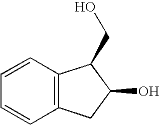

Methyl (1R,2S)-2-hydroxyindane-1-carboxylate (Intermediate 12; 10.56 g, 55.0 mmol) was dissolved in dry THF (100 mL) under a nitrogen atmosphere at 0° C. Lithium borohydride (55.0 mL, 2.0M in THF, 110.0 mmol) was added and the reaction stirred between 0 to 5° C. for 0.5 h, allowed to warm to ambient temperature and stirred for a further 2 h. The mixture was poured into saturated NaHCO₃, extracted with ethyl acetate (200 mL) and the organic phase washed with water (2×50 mL), brine (50 mL) and dried (MgSO₄). The volatiles were removed by evaporation under reduced pressure to give the title compound (9.1 g, 93%) as a colourless oil. ¹HNMR 2.7 (m, 1H), 2.95 (m, 1H), 3.05 (m, 1H), 3.55 (m, 1H), 3.8 (m, 1H), 4.55 (m, 3H), 7.2 (m, 4H).

### Intermediate 12: Methyl (1R,2S)-2-hydroxyindane-1-carboxylate

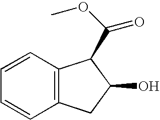

(Reference:Didier, E et al Tetrahedron 47(27), 4941-4958, 1991)

De-ionised water (20 L) was warmed to 34° C., bakers yeast (3 Kg) added and the mixture stirred for 0.5 hr. Methyl 2-oxoindane-1-carboxylate (40 g, 0.21 mmol) was added, the suspension stirred for 3 days and filtered through Celite. The aqueous filtrate was extracted with ethyl acetate (4×2.5 L) and the organic extracts dried (MgSO₄), filtered and the volatiles removed by evaporation under reduced pressure. The crude residues were purified by flash silica gel chromatography (4:1 iso-hexane:ethyl acetate) the solvent evaporated and the resultant solid recrystallised from iso-hexane/ethyl acetate to give the title compound (10.8 g, 27%) as colourless needles.

Mp=72.5-73.5° C. (lit=73.2° C.); [∝]_{D}=+48.7° (C=1.0, CHCl₃) (lit=+48.3°)

¹H NMR 2.85 (dd, 1H), 3.04 (dd, 1H), 3.61 (s, 3H), 4.1 (d, 1H), 4.76 (m, 1H), 5.2 (d, 1H), 7.2 (m, 4H).

### Intermediate 13: tert-Butyl (2R/S)-[((1R,2R)-2-{[(5-chloro-1H-indol-2-yl)carbonyl]amino}-2,3-dihydro-1H-inden-1-yl)methoxy]propanoate

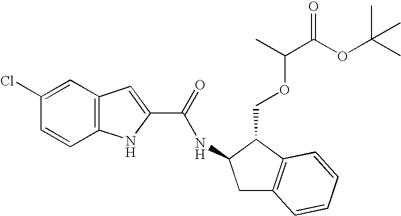

HOBT (185 mg, 1.37 mmol), tert-butyl (2R/S)-{[(1R,2R)-2-amino-2,3-dihydro-1H-inden-1-yl]methoxy}propanoate (Intermediate 15; 400 mg, 1.37 mmol) and EDAC (328 mg, 1.71 mmol) were added to a suspension of 5-chloroindole-2-carboxylic acid (267 mg, 1.37 mmol) in DMA (5 mL). The reaction was stirred at ambient temperature for 20 h. Water (25 mL) was added and the precipitate filtered, washed with water (2×20 mL) and dried. Purification by flash chromatography (SiO₂, iso-hexane:EtOAc, 2:1) gave the title compound (430 mg, 67%) as a foam. ¹H NMR δ: 1.38 (dd, 3H), 1.47 (d, 9H), 3.0 (m, 1H), 3.65 (m, 4.5H), 4.1 (m, 0.5H), 4.56 (m, 1H), 6.82 (dd, 1H), 6.84 (d, 0.5H), 7.22 (m, 5.5H), 7.37 (d, 1H), 7.59 (s, 1H), 9.65 (d, 1H); MS m/z 467/469 (M−H).

The following intermediate was prepared by the method of Intermediate 13, using tert-butyl (2R/S)-{[(1R,2R)-2-amino-2,3-dihydro-1H-inden-1-yl]methoxy}propanoate (Intermediate 15) as the amine and the appropriate carboxylic acid (5-fluoroindole-2-carboxylic acid).

### Intermediate 14: tert-Butyl (2R/S)-[((1R,2R)-2-{[(5-fluoro-1H-indol-2-yl)carbonyl]amino}-2,3-dihydro-1H-inden-1-yl)methoxy]propanoate

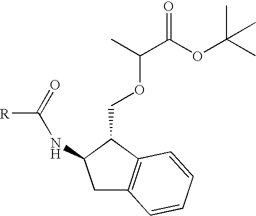

| Intermediate | R | ¹H NMR (CDCl₃) | M/z |
|---|---|---|---|
| 14 | 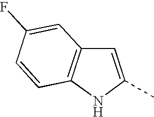 | 1.38 (dd, 3H), 1.45 (d, 9H), 2.99 (m, 1H), 3.67 (m, 4.5H), 4.1 (m, 0.5H), 4.6 (m, 1H), 6.81 (m, 1.5H), 6.9 (d, 0.5H), 7.01 (ddd, 1H), 7.15 (d, 0.5H), 7.23 (m, 3H), 7.35 (m, 2.5H), 9.6 (s, 1H) | 451 (M − H) |

### Intermediate 15; tert-Butyl (2R/S)-{[(1R,2R)-2-amino-2,3-dihydro-1H-inden-1-yl]methoxy}propanoate

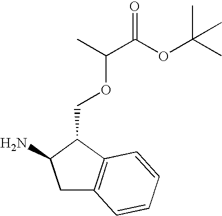

To a solution of tert-butyl (2R/S)-({(1R,2R)-2-[({[tert-butyl(dimethyl)silyl]oxy}carbonyl)amino]-2,3-dihydro-1H-inden-1-yl}methoxy)propanoate (Intermediate 16; 3.1 g, 7.0 mmol) in THF (50 mL) was added tetrabutyl ammoniumfluoride (9.0 mL, 1M in THF, 9.0 mmol) and the reaction stirred at ambient temperature for 4 h. Ammonium chloride solution (25 mL, saturated aqueous) was added and the mixture extracted with EtOAc (2×25 mL). The organic extracts were washed with water (20 mL), brine (20 mL), dried (MgSO₄) and the volatiles removed by evaporation under reduced pressure to give the title compound (1.6 g, 80%) as an oil.

¹H NMR δ (CDCl₃): 1.48 (d, 9H), 3.0 (ddd, 1H), 3.32 (m, 1H), 3.55 (m, 3H), 3.7 (m, 1H), 3.9 (m, 1H), 4.04 (m, 1H), 7.17 (m, 4H); MS m/z 292.

### Intermediate 16: tert-Butyl (2R/S)-({(1R,2R)-2-[({[tert-butyl(dimethyl)silyl]oxy}carbonyl)amino]-2,3-dihydro-1H-inden-1-yl}methoxy)propanoate

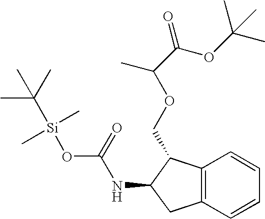

To a solution of tert-butyl (2R/S)-({(1R,2R)-2-[(tert-butoxycarbonyl)amino]-2,3-dihydro-1H-inden-1-yl}methoxy)propanoate (Intermediate 17; 2.75 g, 7.02 mmol) and 2,6-lutidine (1.6 mL, 14.0 mmol) in anhydrous DCM (25 mL) was added tert-butyldimethylsilyl trifluoromethanesulphonate (2.4 mL, 10.54 mmol) and the reaction stirred at ambient temperature for 30 mins. Ammonium chloride solution (20 mL, saturated aqueous) was added and the mixture extracted with EtOAc (2×35 mL). The organic extracts were washed with water (20 mL), brine (20 mL), dried (MgSO₄) and the volatiles removed by evaporation under reduced pressure to give the title compound (3.2 g, 100%) as an oil. MS m/z 472 (M+Na).

### Intermediate 17: tert-Butyl (2R/S)-([(1R,2R)-2-[(tert-butoxycarbonyl)amino]-2,3-dihydro-1H-inden-1-yl]methoxy)propanoate

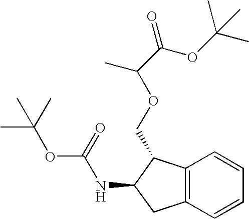

To a solution of tert-butyl[(1R,2R)-1-(hydroxymethyl)-2,3-dihydro-1H-inden-2-yl]carbamate (Intermediate 6: 2.63 g, 10.0 mmol) in DCM (30 mL) was added tert-butyl-(2R/S)-bromo propionate (2.6 g, 12.5 mmol), tetrabutylammonium hydrogen sulphate (850 mg, 2.5 mmol) and sodium hydroxide (9.6 mL, 50% w/v aqueous, 120.0 mmol) and the reaction stirred at ambient temperature for 3 h. Water (50 mL) was added and the mixture extracted with DCM (2×50 mL). The organic extracts were washed with water (25 mL), brine (25 mL), dried (MgSO₄) and the volatiles removed under reduced pressure. The residue was purified by flash chromatography with (SiO₂, iso-hexane:EtOAc, 3:1) gave the title compound (2.5 g, 64%) as an oil.

¹H NMR δ (CDCl₃): 1.42 (m, 21H), 2.78 (ddd, 1H), 3.23 (m, 1H), 3.35 (m, 1H), 3.57 (m, 1H), 4.85 (m, 2H), 4.14 (m, 1H), 4.9 (m, 1H), 7.17 (m, 3H), 7.37 (m, 1H); MS m/z 414 (M+Na).

### Intermediate 18: Methyl ((1R,2R)-2-{[(5-fluoro-1H-indol-2-yl)carbonyl]amino}-2,3-dihydro-1H-inden-1-yl)acetate

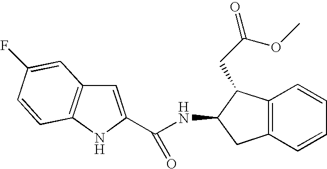

5-Fluoroindole-2-carboxylic acid (75 mg, 0.42 mmol), methyl[(1R,2R)-2-amino-2,3-dihydro-1H-inden-1-yl]acetate hydrochloride salt (Intermediate 20, 113 mg, 0.42 mmol), DIPEA (144 μL, 0.84 mmol) and HOBT (57 mg, 0.42 mmol) were dissolved in DMA (5 mL). EDAC (102 mg, 0.53 mmol) was added and the reaction stirred at ambient temperature for 19 h. The volatiles were removed under reduced pressure and the crude material dissolved in EtOAc (15 mL). The organic phase was washed with water (3×15 mL), brine (15 mL), dried (MgSO₄) and the solvent removed in vacuo. Purification by flash column chromatography (SiO₂, 1:5 EtOAc:hexanes to 3:2 EtOAc:hexanes gradient) afforded the title compound (100 mg, 65%) as a solid.

¹H-NMR δ: 2.73 (m, 2H), 2.93 (dd, 1H), 3.25 (dd, 1H), 3.57 (s, 3H), 3.63 (m, 1H), 4.48 (m, 1H), 7.02 (t, 1H), 7.17 (m, 5H), 7.4 (m, 2H), 8.70 (d, 1H), 11.63 (s, 1H); MS m/z 367.

The following example was made by the process of Intermediate 18 using methyl [(1R,2R)-2-amino-2,3-dihydro-1H-inden-1-yl]acetate hydrochloride salt (Intermediate 20) and 5-chloroindole-2-carboxylic acid as starting material:

### Intermediate 19: Methyl ((1R,2R)-2-{[(5-chloro-1H-indol-2-yl)carbonyl]amino}-2,3-dihydro-1H-inden-1-yl)acetate

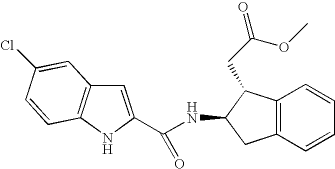

¹H NMR δ: 2.73 (d, 2H), 2.93 (dd, 1H), 3.25 (dd, 1H), 3.57 (s, 3H), 3.63 (m, 1H), 4.48 (m, 1H), 7.17 (m, 6H), 7.42 (d, 1H), 7.68 (s, 1H), 8.72 (d, 1H), 11.73 (s, 1H); MS m/z 383.

### Intermediate 20: Methyl[(1R,2R)-2-amino-2,3-dihydro-1H-inden-1-yl]acetate hydrochloride

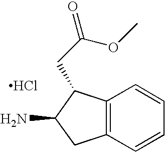

Methyl {(1R,2R)-2-[(tert-butoxycarbonyl)amino]-2,3-dihydro-1H-inden-1-yl}acetate (Intermediate 21; 4.09 g, 13 mmol) was dissolved in DCM (20 mL) and treated with HCl (20 mL, 4M in dioxane) and stirred at ambient temperature for 1 h. Volatiles were then removed by evaporation under reduced pressure. The resulting white solid was stirred with ether (70 mL) and recovered by filtration to give the title compound (2.96 g, 91%).

¹H NMR δ: 2.73 (m, 1H), 2.99 (m, 2H), 3.31 (m, 1H), 3.60 (m, 4H), 3.76 (m, 1H), 7.18 (m, 4H), 8.51 (s, 3H); MS m/z 206.

### Intermediate 21: Methyl {(1R,2R)-2-[(tert-butoxycarbonyl)amino]-2,3-dihydro-1H-inden-1-yl}acetate

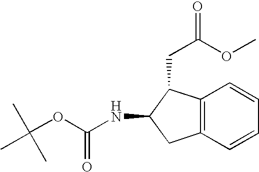

Sodium chloride (405 mg, 6.93 mmol) was added to a solution of dimethyl {(1R,2R)-2-[(tert-butoxycarbonyl)amino]-2,3-dihydro-1H-inden-1-yl}malonate (Intermediate 22; 630 mg, 1.73 mmol) in DMSO (8 mL) containing 4 drops of water and the reaction was heated to 160° C. for 46 h. The solvent was removed on a Genevac EZ-2 centrifugal evaporator and the residue was taken up in water (25 mL) and EtOAc (25 mL). The organic layer was dried (MgSO₄), filtered and evaporated. Purification by column chromatography (SiO₂, EtOAc:hexanes, 1:2) afforded the title compound (360 mg, 68%) as a solid.

¹H NMR δ: 1.45 (s, 9H), 2.78 (m, 2H), 3.38 (m, 2H), 3.75 (s, 3H), 4.13 (m, 1H), 4.87 (br. s, 1H), 7.17 (m, 4H); MS m/z 386 [M+Na+MeCN]⁺.

### Intermediate 22: Dimethyl {(1R,2R)-2-[(tert-butoxycarbonyl)amino]-2,3-dihydro-1H-inden-1-yl}malonate

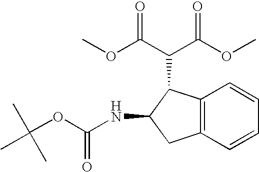

NaHMDS (6 mL, 1 M in THF, 6.00 mmol) was added to a stirred solution of (1S,2S)-1-[(tert-Butoxycarbonyl)amino]-2,3-dihydro-1H-inden-2-yl methanesulfonate (Intermediate 23, 1.79 g, 5.46 mmol) in THF (24 mL) whilst keeping the internal temperature <20° C. After 30 mins dimethyl malonate (0.69 mL, 6.00 mmol) was added followed by NaHMDS (6 mL, 1 M in THF, 6.00 mmol) and the reaction was heated at 50° C. for 18.5 h. The reaction was cooled (ambient temperature) and quenched with ammonium chloride solution (50 mL, saturated aqueous) and Et₂O (50 ml) and the aqueous layer was re-extracted with Et₂O (50 mL). The combined organic extracts were dried (MgSO₄), filtered and the volatiles removed in vacuo. Purification by flash column chromatography (SiO₂, eluent gradient: 1:3 to 1:1 EtOAc:hexanes) afforded the title compound (630 mg, 32%) as a white solid.

¹H NMR δ: 1.45 (s, 9H), 2.78 (dd, 1H), 3.37 (dd, 1H), 3.72 (m, 8H), 4.40 (m, 1H), 4.78 (br. s, 1H), 7.20 (m, 4H); MS m/z 386 [M+Na].

### Intermediate 23: (1S,2S)-1-[(tert-Butoxycarbonyl)amino]-2,3-dihydro-1H-inden-2-yl methanesulfonate

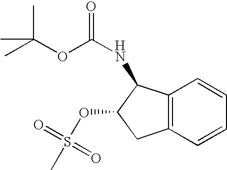

Mesyl chloride (2.24 mL, 30.03 mmol) was added to a cooled (0° C.) solution of tert-butyl [(1S,2S)-2-hydroxy-2,3-dihydro-1H-inden-1-yl]carbamate (Intermediate 24, 6.80 g, 27.3 mmol) and triethylamine (4.01 mL, 30.03 mmol) in DCM (100 mL) and stirred at 0° C. for 1 h. The reaction was quenched by addition of saturated aqueous NaHCO₃ (100 mL), the organic layer was dried (MgSO₄), filtered and the volatiles removed in vacuo. The crude product was triturated with hot Et₂O (40 mL), cooled and filtered to afford the title compound (8.11 g, 91%) as a white solid.

¹H NMR δ: 1.45 (s, 9H), 3.18 (m, 4H), 3.47 (dd, 1H), 4.78 (s, 1H) 5.19 (m, 2H), 7.28 (m, 4H); MS m/z 350 [M+Na]⁺.

### Intermediate 24: tert-Butyl[(1S,2S)-2-hydroxy-2,3-dihydro-1H-inden-1-yl]carbamate

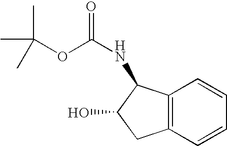

THF (100 mL) followed by 1 M aqueous sodium hydroxide was added to (1S,2S)(+)-trans-1-amino-2-indanol (CAS Reg. No. 163061-74-3, 5.00 g, 33.55 mmol). Di-tert-butyl dicarbonate (7.30 g, 33.55 mmol) was then added and stirred for 16 h. The THF was removed in vacuo and the remaining aqueous layer was acidified to pH 2 with citric acid (5% w/v aq.) and diluted with EtOAc (150 mL). The organic layer was dried (MgSO₄), filtered and the volatiles removed in vacuo. The crude was triturated with hot Et₂O:hexanes (1:1, 40 mL), the suspension cooled and filtered to afford the title compound (6.80 g, 81%) as a white solid.

¹H NMR δ: 1.54 (s, 9H), 2.92 (dd, 1H), 3.28 (dd, 1H), 4.23 (s, 1H), 4.42 (m, 1H), 4.93 (t, 1H), 5.03 (s, 1H), 7.22 (m, 4H); MS m/z 313 [M+Na+MeCN]⁺.

### Intermediate 25: Methyl ((1R,2R)-2-{[(5,6-difluoro-1H-indol-2-yl)carbonyl]amino}-2,3-dihydro-1H-inden-1-yl)acetate

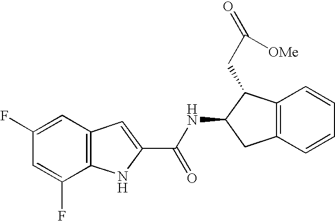

5,6-Difluoro-1H-indole-2-carboxylic acid (Intermediate 26, 98.5 mg, 0.5 mmol), methyl [(1R,2R)-2-amino-2,3-dihydro-1H-inden-1-yl]acetate hydrochloride (Intermediate 20, 121 mg, 0.5 mmol), DIPEA (193.5 mg, 1.5 mmol) and HOBT (81 mg, 0.6 mmol) were dissolved in DMF (2 mL). EDAC (114.6 mg, 0.6 mmol) was added and the mixture stirred at ambient temperature for 6 h.

Ethyl acetate was added (50 mL) and the mixture washed with water (2×10 mL), 2M HCl (10 mL), saturated sodium bicarbonate (10 mL) and brine (10 mL). After drying (MgSO₄) the volatiles were removed by evaporation under reduced pressure and the residue purified by flash column chromatography using a 0-20% ethyl acetate/DCM gradient to give the title compound (132 mg, 68%) as a white solid. MS m/z 383 (m-H)⁻

### Intermediate 26: 5,6-Difluoro-1H-indole-2-carboxylic acid

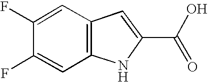

4,5-Difluoro-2-iodoaniline (Intermediate 27, 1.24 g, 4.86 mmol) was dissolved in anhydrous DMF (15 mL). Pyruvic acid (1.32 g, 15 mmol) and DABCO (1.68 g, 15 mmol) were added and the mixture degassed by alternate vacuum and nitrogen purge. Palladium acetate (56 mg) was then added and the mixture heated to 100° C. for 2 h. After cooling to ambient temperature the reaction mixture was filtered through celite, diluted with ethyl acetate (100 mL), washed with 2M HCl (2×20 mL) and water (2×20 mL), dried (MgSO₄) and evaporated to give the title compound as a dark solid (860 mg, 90%).

¹H NMR δ: 7.0 (s, 1H), 7.3 (dd, 1H), 7.6 (dd, 1H), 11.9 (s, 1H); MS m/z 196 (m-H)⁻.

### Intermediate 27: 4,5-Difluoro-2-iodoaniline

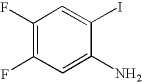

3,4-Difluoroaniline (645 mg, 5 mmol) was suspended in water (25 mL). Sodium bicarbonate (630 mg, 7.5 mmol) was added and then iodine (1.65 g, 6.5 mmol). The reaction mixture was stirred vigorously at ambient temperature for 30 min and then poured into saturated sodium thiosulphate solution (50 mL) and extracted with ethyl acetate (2×25 mL). The combined extracts were washed with sodium thiosulphate (20 mL), water (20 mL) and brine (20 mL), dried (MgSO₄) and evaporated to give the title compound as a dark oil. (1.24 g, 97%).

### Intermediate 28: {(1R,2R)-2-[(5-Chloro-7-fluoro-1H-indole-2-carbonyl)-amino]-indan-1-yl}-acetic acid methyl ester

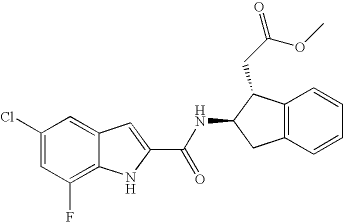

5-Chloro-7-fluoro-1H-indole-2-carboxylic acid (Intermediate 29, 213 mg, 1 mmol), ((1R,2R)-2-amino-indan-1-yl)-acetic acid methyl ester hydrochloride (240 mg, 1 mmol), DIPEA (347 μL, 2 mmol) and HOBT (135 mg, 1 mmol) were dissolved in DMF (10 mL). EDCI (238 mg, 1.25 mmol) was then added and the mixture stirred at ambient temperature for 18 h. The reaction mixture was diluted with ethyl acetate (100 mL), washed with water (3×25 mL) and brine (25 mL), dried (MgSO₄) and evaporated to leave an oil which was purified by chromatography on silica gel (EtOAc/DCM 0-15%) to give the title compound as a foam. (202 mg, 50%). ¹H NMR (400 MHz, DMSO-d₆) δ 2.75 (2H, d), 2.90-2.96 (1H, m), 3.25-3.35 (1H, m), 3.57 (3H, s), 3.64 (1H, t), 4.46-4.53 (1H, m), 7.16-7.26 (6H, m), 7.59 (1H, s), 8.77 (1H, d), 12.25 (1H, s); MS m/z 401

### Intermediate 29: 5-Chloro-7-fluoro-1H-indole-2-carboxylic acid

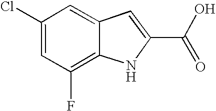

4-Chloro-2-fluoro-6-iodoaniline (Intermediate 30, 1.35 g, 5 mmol), pyruvic acid (1.32 g, 15 mmol) and DABCO (1.68 g, 15 mmol) were dissolved in DMF (15 mL). The solution was degassed and palladium acetate (56 mg) was added. The mixture was then heated to 100° C. for 3 h., cooled to ambient and filtered. The filtrate was diluted with EtOAc (100 mL), washed with 2M HCl (2×20 mL), water (20 mL) and brine (20 mL), dried (MgSO₄) and evaporated to leave the title compound as a dark solid (0.9 g, 85%). ¹H NMR (400 MHz, DMSO-d₆) δ 7.16 (1H, t), 7.21-7.24 (1H, m), 7.59 (1H, d), 12.52 (1H, s), 13.27 (1H, s); MS m/z 212 (M−H)⁻

### Intermediate 30: 4-Chloro-2-fluoro-6-iodoaniline

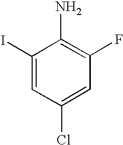

4-Chloro-2-fluoroaniline (2.9 g, 20 mmol) was dissolved in ethanol (200 mL). Silver sulphate (6.22 g, 20 mmol) was added and then iodine (5.08 g, 20 mmol) was added in small portions. After the addition was complete the reaction mixture was stirred at ambient temperature for 90 min. The reaction mixture was filtered through Celite and evaporated to leave a dark oil which was taken up in DCM (200 mL). and washed with 2M sodium hydroxide (2×50 mL), saturated sodium thiosulphate (2×50 mL) and water (2×50 mL). The solution was dried (MgSO₄) and evaporated to leave the title compound as a dark oil (4.73 g, 87%).

¹H NMR (400 MHz, DMSO-d₆) δ 5.30 (2H, s), 7.25-7.29 (1H, m), 7.47 (1H, t)

### Intermediate 31: 2-{(1S,2R)-2-[(5-Chloro-1H-indole-2-carbonyl)-amino]-indan-1-yl}-2-(2-methoxy-ethyl)-malonic acid diethyl ester

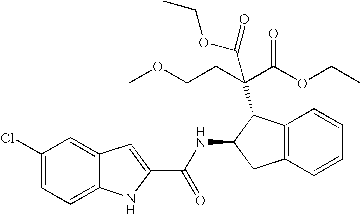

5-Chloro-1H-indole-2-carboxylic (109 mg, 0.55 mmol), 2-((1S,2R)-2-Amino-indan-1-yl)-2-(2-methoxy-ethyl)-malonic acid diethyl ester hydrochloride (Intermediate 32, 160 mg, 0.51 mmol), DIPEA (0.11 mL, 0.31 mmol) and HOBT (99.5 mg, 0.51 mmol) were dissolved in DCM (10 mL). EDAC (122 g, 0.64 mmol) was added and the reaction stirred at ambient temperature for 19 h. DCM was evaporated under reduced pressure, water (10 mL) was added then the mixture extracted with EtOAc (2×20 mL). The organic phases were combined and washed with saturated aqueous sodium bicarbonate (10 mL), water (10 mL), dried (MgSO₄) and the solvent was removed in vacuo to afford the title compound (200 mg, 75%) as an oil. MS m/z 526.

### Intermediate 32: 2-((1S,2R)-2-Amino-indan-1-yl)-2-(2-methoxy-ethyl)-malonic acid diethyl ester hydrochloride

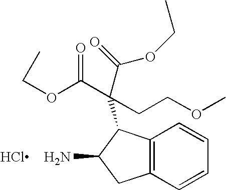

2-((1S,2R)-2-tert-Butoxycarbonylamino-indan-1-yl)-2-(2-methoxy-ethyl)-malonic acid diethyl ester (Intermediate 33, 383 mg, 3.89 mmol) was treated with HCl (20 mL, 4M in dioxane) and stirred at ambient temperature for 2 h. Volatiles were then removed by evaporation under reduced pressure and the product further dried under vacuum) to give the title compound as an oil (300 mg, 100%).

### Intermediate 33: 2-((1S,2R)-2-tert-Butoxycarbonylamino-indan-1-yl)-2-(2-methoxy-ethyl)-malonic acid diethyl ester

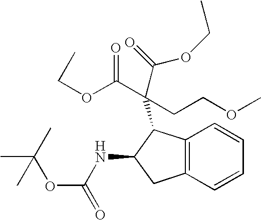

NaHMDS (3.3 mL, 1 M in THF, 3.3 mmol) was added to a stirred solution of (1S,2S)-1-[(tert-Butoxycarbonyl)amino]-2,3-dihydro-1H-inden-2-yl methanesulfonate (Intermediate 34, 1 g, 3 mmol) in THF (30 mL) whilst keeping the internal temperature <5° C. After 30 mins diethyl (2-methoxyethyl)malonate (720 mg, 3.3 mmol) was added followed by NaHMDS (1.65 mL, 1 M in THF, 1.65 mmol) and the reaction was allowed to warm at room temperature and stirred for 18 h. The reaction was quenched with saturated aqueous ammonium chloride solution (50 mL) and Et₂O (50 mL) and the aqueous layer was re-extracted with Et₂O (50 mL). The combined organic extracts were dried (MgSO₄), filtered and the volatiles removed in vacuo. Purification by flash column chromatography (SiO₂, eluent gradient: 0% to 40% hexanes:EtOAc) afforded the title compound (546 mg, 1.21 mmol, 40%) as an oil. MS m/z 450

### Intermediate 34: (1S,2S)-1-[(tert-Butoxycarbonyl)amino]-2,3-dihydro-1H-inden-2-yl methanesulfonate

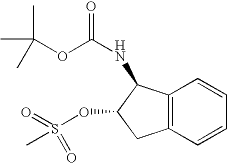

Mesyl chloride (2.24 mL, 30.03 mmol) was added to a cooled (0° C.) solution of tert-butyl [(1S,2S)-2-hydroxy-2,3-dihydro-1H-inden-1-yl]carbamate (Intermediate 35, 6.80 g, 27.3 mmol) and triethylamine (4.01 mL, 30.03 mmol) in DCM (100 mL) and stirred at 0° C. for 1 h. The reaction was quenched by addition of saturated aqueous sodium bicarbonate (100 mL), the organic layer was dried (MgSO₄), filtered and the volatiles removed in vacuo. The crude product was triturated with hot Et₂O (40 mL), cooled and filtered to afford the title compound (8.11 g, 91%) as a white solid. ¹H NMR δ: 1.45 (s, 9H), 3.18 (m, 4H), 3.47 (dd, 1H), 4.78 (s, 1H) 5.19 (m, 2H), 7.28 (m, 4H); MS m/z 350 [M+Na]⁺.

### Intermediate 35: tert-Butyl[(1S,2S)-2-hydroxy-2,3-dihydro-1H-inden-1-yl]carbamate

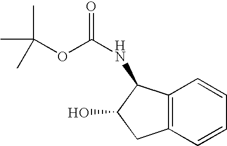

THF (100 mL) followed by 1 M aqueous sodium hydroxide was added to (1S,2S)(+)-trans-1-amino-2-indanol (CAS Reg. No. 163061-74-3, 5.00 g, 33.55 mmol). Di-tert-butyl dicarbonate (7.30 g, 33.55 mmol) was then added and stirred for 16 h. The THF was removed in vacuo and the remaining aqueous layer was acidified to pH 2 with citric acid (5% w/v aqueous) and diluted with EtOAc (150 mL). The organic layer was dried (MgSO-₄), filtered and the volatiles removed in vacuo. The crude solid was triturated with hot Et₂O:hexanes (1:1, 40 mL), the suspension cooled and filtered to afford the title compound (6.80 g, 81%) as a white solid.

¹H NMR δ: 1.54 (s, 9H), 2.92 (dd, 1H), 3.28 (dd, 1H), 4.23 (s, 1H), 4.42 (m, 1H), 4.93 (t, 1H), 5.03 (s, 1H), 7.22 (m, 4H); MS m/z 313 [M+Na+MeCN]⁺.

### Intermediate 36: 2-{(1R,2R)-2-[(5-Chloro-1H-indole-2-carbonyl)-amino]-indan-1-yl}-propionic acid methyl ester

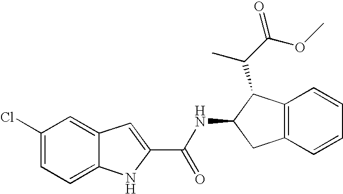

5-Chloro-1H-indole-2-carboxylic (69 mg, 0.35 mmol), 2-((1R,2R)-2-Amino-indan-1-yl)-propionic acid methyl ester hydrochloride salt (Intermediate 37, 80 mg, 0.32 mmol), DIPEA (0.07 mL, 0.38 mmol) and HOBT (43 mg, 0.32 mmol) were dissolved in DCM (10 mL). EDAC (77 mg, 0.40 mmol) was added and the reaction stirred at ambient temperature for 19 h. The volatiles were removed under reduced pressure and the crude material dissolved in EtOAc (15 mL). The organic phase was washed with a solution of saturated bicarbonate (10 mL), water (10 mL), brine (10 mL), dried (MgSO₄) and the solvent removed in vacuo to give the title compound (120 mg, 94%) as a solid. MS m/z 419 [M+Na].

### Intermediate 37: 2-((1R,2R)-2-Amino-indan-1-yl)-propionic acid methyl ester hydrochloride

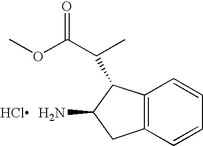

2-((1R,2R)-2-tert-Butoxycarbonylamino-indan-1-yl)-propionic acid methyl ester (Intermediate 38; 200 mg, 0.63 mmol) was treated with HCl (20 mL, 4M in dioxane) and stirred at ambient temperature for 2 h. Volatiles were then removed by evaporation under reduced pressure to give the title compound (164 mg, 100%). MS m/z 220.

### Intermediate 38: 2-((1R,2R)-2-tert-Butoxycarbonylamino-indan-1-yl)-propionic acid methyl ester

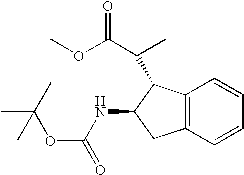

Sodium chloride (251 mg, 4.4 mmol) was added to a solution of 2-((1S,2R)-2-tert-Butoxycarbonylamino-indan-1-yl)-2-methyl-malonic acid dimethyl ester (Intermediate 39; 630 mg, 1.73 mmol) in DMSO (5 mL) containing 4 drops of water and the reaction was heated to 160° C. for 4 h. The reaction mixture was taken up in water (25 mL) and EtOAc (25 mL). The organic layer was washed twice with water (10 mL), dried (MgSO₄), filtered and evaporated under reduced pressure. Purification by column chromatography (SiO₂, eluent gradient: 0% to 30% hexanes:EtOAc) afforded the title compound (200 mg, 57%) as a yellow oil. MS m/z 342 [M+Na].

### Intermediate 39: 2-((1S,2R)-2-tert-Butoxycarbonylamino-indan-1-yl)-2-methyl-malonic Acid Dimethyl Ester

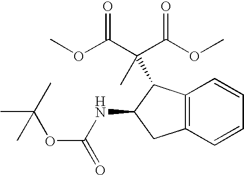

NaHMDS (1.65 mL, 1 M in THF, 1.65 mmol) was added to a stirred solution of (1S,2S)-1-[(tert-butoxycarbonyl)amino]-2,3-dihydro-1H-inden-2-yl methanesulfonate (Intermediate 40, 0.5 g, 1.5 mmol) in THF (15 mL) whilst keeping the internal temperature <5° C. After 30 mins dimethyl (2-methyl)malonate (0.22 mL, 1.65 mmol) was added followed by NaHMDS (0.83 mL, 1 M in THF, 0.83 mmol) and the reaction was allowed to warm at room temperature and stirred for 18 h. The reaction was quenched with water (20 mL) and Et₂O (40 ml) and the aqueous layer was re-extracted with Et₂O (15 mL). The combined organic extracts were successively washed with a solution of saturated bicarbonate (20 mL), brine (20 mL). The organic phase was dried (MgSO₄), filtered and the volatiles removed in vacuo. Purification by flash column chromatography (SiO₂, eluent gradient: 0% to 40% hexanes:EtOAc) afforded the title compound (424 mg, 75%) as a white gum. MS m/z 400 [M+Na].

### Intermediate 40: (1S,2S)-1-[(tert-Butoxycarbonyl)amino]-2,3-dihydro-1H-inden-2-yl Methanesulfonate

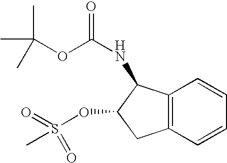

Mesyl chloride (2.24 mL, 30.03 mmol) was added to a cooled (0° C.) solution of tert-butyl [(1S,2S)-2-hydroxy-2,3-dihydro-1H-inden-1-yl]carbamate (Intermediate 41, 6.80 g, 27.3 mmol) and triethylamine (4.01 mL, 30.03 mmol) in DCM (100 mL) and stirred at 0° C. for 1 h. The reaction was quenched by addition of saturated aqueous sodium bicarbonate (100 mL), the organic layer was dried (MgSO₄), filtered and the volatiles removed in vacuo. The crude product was triturated with hot Et₂O (40 mL), cooled and filtered to afford the title compound (8.11 g, 91%) as a white solid. ¹H NMR δ: 1.45 (s, 9H), 3.18 (m, 4H), 3.47 (dd, 1H), 4.78 (s, 1H) 5.19 (m, 2H), 7.28 (m, 4H); MS m/z 350 [M+Na]⁺.

### Intermediate 41: tert-Butyl[(1S,2S)-2-hydroxy-2,3-dihydro-1H-inden-1-yl]carbamate

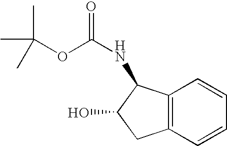

THF (100 mL) followed by 1 M sodium hydroxide (aqueous) was added to (1S,2S)(+)-trans-1-amino-2-indanol (CAS Reg. No. 163061-74-3, 5.00 g, 33.55 mmol). Di-tert-butyl dicarbonate (7.30 g, 33.55 mmol) was then added and stirred for 16 h. The THF was removed in vacuo and the remaining aqueous layer was acidified to pH 2 with citric acid (5% w/v aq.) and diluted with EtOAc (150 mL). The organic layer was dried (MgSO₄), filtered and the volatiles removed in vacuo. The crude solid was triturated with hot Et₂O:hexanes (1:1, 40 mL), the suspension cooled and filtered to afford the title compound (6.80 g, 81%) as a white solid. ¹H NMR δ: 1.54 (s, 9H), 2.92 (dd, 1H), 3.28 (dd, 1H), 4.23 (s, 1H), 4.42 (m, 1H), 4.93 (t, 1H), 5.03 (s, 1H), 7.22 (m, 4H); MS m/z 313 [M+Na+MeCN]⁺.

1. A compound of formula (1): 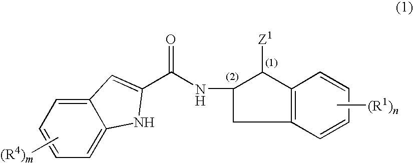
wherein:
n is 0, 1 or 2;
m is 0, 1 or 2;
R¹ is independently selected from halo, nitro, cyano, hydroxy, carboxy, carbamoyl, N-(1-4C)alkylcarbamoyl, N,N-((1-4C)alkyl)₂-carbamoyl, sulphamoyl, N-(1-4C)alkylsulphamoyl, N,N-((1-4C)alkyl)₂sulphamoyl, (1-4C)alkylS(O)_{b} (wherein b is 0, 1, or 2), —OS(O)₂(1-4C)alkyl, (1-4C)alkyl, (2-4C)alkenyl, (2-4C)alkynyl, (1-4C)alkoxy, (1-4C)alkanoyl, (1-4C)alkanoyloxy, hydroxy(1-4C)alkyl, fluoromethyl, difluoromethyl, trifluoromethyl, trifluoromethoxy and —NHSO₂(1-4C)alkyl;
or, when n is 2, the two R¹ groups, together with the carbon atoms to which they are attached, may form a 4 to 7 membered saturated ring, optionally containing 1 or 2 heteroatoms independently selected from O, S and N, and optionally being substituted by one or two methyl groups;
R⁴ is independently selected from halo, nitro, cyano, hydroxy, fluoromethyl, difluoromethyl, trifluoromethyl, trifluoromethoxy, carboxy, carbamoyl, (1-4C)alkyl, (2-4C)alkenyl, (2-4C)alkynyl, (1-4C)alkoxy and (1-4C)alkanoyl;
Z¹ is either
a) of the formula —Y—COOH wherein Y is (1-6C)alkylene or (3-6C)cycloalkylene; or
b) of the formula —Y—COOH; wherein Y is (1-6C)allylene which is:
ii) interrupted by one heteroatom selected from —N(R⁷)—, —O—, —S—, —SO— and —SO₂-(provided that the heteroatom is not adjacent to the carboxy group and wherein R⁷ is hydrogen, (1-4C)alkyl, (1-4C)alkanoyl or (1-4C)alkylsulphonyl); and/or
ii) substituted on carbon by 1 or 2 substituents independently selected from cyano, oxo, hydroxyl, (1-3C)alkoxy, (1-3C)alkanoyl, (1-3C)alkoxy(2-3C)alkoxy, hydroxy(1-3C)alkyl, hydroxy(2-3C)alkoxy, (3-6C)cycloalkyl, (3-6C)cycloalkyl(1-3C)alkyl, (3-6C)cycloalkyloxy, (3-6C)cycloalkyl(1-3C)alkoxy, (1-3C)alkylS(O)_{c} (wherein c is 0, 1 or 2), —CON(R²)R³, —N(R²)COR³, —SO₂N(R²)R³ and —N(R²)SO₂R³ wherein R² and R³ are independently selected from hydrogen and (1-3C)alkyl;
or when the alkylene group is interrupted by one heteroatom it may also be optionally substituted on a carbon by 2 substituents which together with the carbon atom to which they are attached form a (3-6C)cycloalkyl ring;
or a pharmaceutically acceptable salt thereof.

2. A compound of formula (1) as claimed in claim 1, or a pharmaceutically-acceptable salt thereof wherein m is 1 or 2 and each R⁴ is independently Cl or F.

3. A compound of formula (1) as claimed in claim 1 or claim 2, or a pharmaceutically-acceptable salt thereof wherein n=0.

4. A compound of formula (1) as claimed in any one of claims 1 to 3, or a pharmaceutically-acceptable salt thereof, wherein Y is selected from option a).

5. A compound of formula (1) as claimed in any one of claims 1 to 3, or a pharmaceutically-acceptable salt thereof, wherein Y is selected from option b).

6. A compound of formula (1) as claimed in claim 5, or a pharmaceutically-acceptable salt thereof, wherein Y is selected from —CH₂XCH₂—, —CH₂XCH₂CH₂—, —CH₂CH₂XCH₂, —CH(R^{f})XCH₂—, —CH(R^{f})XCH₂CH₂—, —CH(R^{f})CH₂XCH₂—, —CH₂CH(R)XCH₂—, —CH₂CH₂XCH(R^{f})—, —CH₂XCH(R^{f})CH₂—, —CH₂XCH(R^{f})—, —CH₂XCR^{f}₂—, —CH₂XCH₂CH₂CH₂— [wherein X is selected from —O—, —S— and —SO₂— and R^{f} is selected from methyl and ethyl], —CH₂—, —CH₂CH₂—, —CH₂CH₂CH₂—, —CH₂CH(Me)—, —CH(R^{g})— and —CH(R^{g})CH₂— [wherein R^{g} is selected from methoxymethyl, ethoxyethyl, methoxyethyl, ethoxymethyl, methoxypropyl, cyclopropylmethyl, isopropylmethyl, ethyl and propyl].

7. A compound of formula (1) as claimed in claim 5 or claim 6, or a pharmaceutically-acceptable salt thereof, wherein Y is selected from —CH₂—, —CH(CH₃)—, —CH₂OCH₂—,
—CH₂OCH(CH₃)— and —CH(CH₂CH₂OCH₃)—.

8. A compound of formula (1) as claimed in claim 4 or a pharmaceutically-acceptable salt thereof, wherein Y is (1-6C)allylene.

9. A compound of formula (1) as claimed in claim 1 or a pharmaceutically-acceptable salt thereof, which is any one or more of the following:
[((1R,2R)-2-{[(5-chloro-1H-indol-2-yl)carbonyl]amino}-2,3-dihydro-1H-inden-1-yl)methoxy]acetic acid;
[((1R,2R)-2-{[(5-fluoro-1H-indol-2-yl)carbonyl]amino}-2,3-dihydro-1H-inden-1-yl)methoxy]acetic acid;
(2R/S)-[((1R,2R)-2-{[(5-chloro-1H-indol-2-yl)carbonyl]amino}-2,3-dihydro-1H-inden-1-yl)methoxy]propanoic acid;
(2R/S)-[((1R,2R)-2-{[(5-fluoro-1H-indol-2-yl)carbonyl]amino}-2,3-dihydro-1H-inden-1-yl)methoxy]propanoic acid;
((1R,2R)-2-{[(5-fluoro-1H-indol-2-yl)carbonyl]amino}-2,3-dihydro-1H-inden-1-yl)acetic acid;
((1R,2R)-2-{[(5-chloro-1H-indol-2-yl)carbonyl]amino}-2,3-dihydro-1H-inden-1-yl)acetic acid
((1R,2R)-2-{[(5,6-difluoro-1H-indol-2-yl)carbonyl]amino}-2,3-dihydro-1H-inden-1-yl)acetic acid;
{(1R,2R)-2-[(5-chloro-7-fluoro-1H-indole-2-carbonyl)-amino]-indan-1-yl}-acetic acid;
(2R)-2-{(1R,2R)-2-[(5-chloro-1H-indole-2-carbonyl)-amino]-indan-1-yl}-4-methoxy-butyric acid;
(2S)-2-{(1R,2R)-2-[(5-chloro-1H-indole-2-carbonyl)-amino]-indan-1-yl}-4-methoxy-butyric acid;
(2R)-2-{(1R,2R)-2-[(5-chloro-1H-indole-2-carbonyl)-amino]-indan-1-yl}-propionic acid; and
(2S)-2-{(1R,2R)-2-[(5-chloro-1H-indole-2-carbonyl)-amino]-indan-1-yl}-propionic acid.

10. A pharmaceutical composition which comprises a compound of the formula (1), or a pharmaceutically acceptable salt thereof, as claimed in claim 1 in association with a pharmaceutically-acceptable diluent or carrier.

11. A compound of the formula (1), or a pharmaceutically acceptable salt thereof, as claimed in claim 1, for use in a method of treatment of a warm-blooded animal such as man by therapy.

12. A compound of the formula (1), or a pharmaceutically acceptable salt thereof, as claimed in claim 1, for use as a medicament.

13. A compound of the formula (1), or a pharmaceutically acceptable salt thereof, as claimed in claim 1, for use as a medicament in the treatment of type 2 diabetes, insulin resistance, syndrome X, hyperinsulinaemia, hyperglucagonaemia, cardiac ischaemia or obesity in a warm-blooded animal such as man.

14. The use of a compound of the formula (1), or a pharmaceutically acceptable salt thereof, as claimed in claim 1, in the manufacture of a medicament for use in the treatment of type 2 diabetes, insulin resistance, syndrome X, hyperinsulinaemia, hyperglucagonaemia, cardiac ischaemia or obesity in a warm-blooded animal such as man.

15. The use of a compound of the formula (1), or a pharmaceutically acceptable salt thereof, as claimed in claim 1, in the manufacture of a medicament for use in the treatment of type 2 diabetes in a warm-blooded animal such as man.

16. A process for preparing a compound of formula (1) as claimed in claim 1, or a pharmaceutically acceptable salt thereof which process (wherein Z₁, Y, R¹, R⁴, m, and n are, unless otherwise specified, as defined in formula (1)) comprises of:
a) reacting an acid of the formula (2): 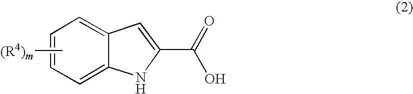
or an activated derivative thereof; with an amine of formula (3): 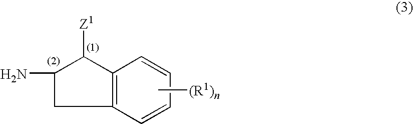
and thereafter if necessary:
i) converting a compound of the formula (1) into another compound of the formula (1);
ii) removing any protecting groups;
iii) forming a pharmaceutically acceptable salt.
